(19) United States
(12) Patent Application Publication
(10) Pub. No.: US 2004/0063686 A1
(43) Pub. Date: Apr. 1, 2004

(54) Carboxamide compounds and their use as antagonists of a human 11cby receptor

(76) Inventors: Christopher Norbert Johnson, Harlow (GB); Martin Jones, Harlow (GB); Catherine Anne O'Toole, Brentwood (GB); Geoffrey Stemp, Harlow (GB); Kevin Michael Thewlis, Harlow (GB); David Witty, Harlow (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/343,424

(22) PCT Filed: Jul. 26, 2001
(86) PCT No.: PCT/EP01/08637

(30) Foreign Application Priority Data
Jul. 31, 2000 (GB) 0018758.3

(51) Int. Cl. A61K 31/553; A61K 31/554; A61K 31/55; A61K 31/541; A61K 31/5377; A61K 31/496; A61K 31/454; A61K 31/427; A61K 31/422; A61K 31/4178; A61K 31/4025

(52) U.S. Cl. 514/211.15; 514/217.03; 514/235.5; 514/252.13; 514/326; 514/365; 514/374; 514/397; 514/422

(57) ABSTRACT

Compounds of formula (I) in which: each A is independently hydrogen, C1-6alkyl optionally substituted by hydroxyl, C1-6alkoxy, C1-6alkenyl or C1-6acyl group or a halogen atom or hydroxyl, CN or CF3 group; R3 is hydrogen, methyl or ethyl; R4 is an optionally substituted aromatic carbocyclic or heterocyclic ring; Z is an O or S atom, or an NH or CH2 group, or a single bond, at the 3 or 4 position of R4 relative to the carbonyl group; R5 is an optionally substituted aromatic carbocyclic or heterocyclic ring, or an optionally substituted, saturated or unsaturated, carbocyclic or heterocyclic ring; and Q is (a) Where X, Y, R1 and R2 are as defined in claim 1; are antagonists of a human 11CBy receptor.

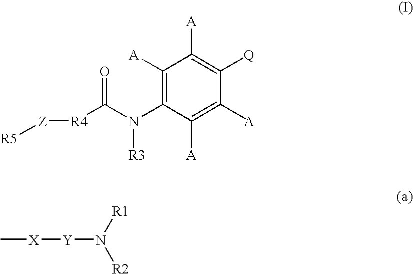

This invention relates to a method of treatment using an antagonist of the human 11CBy receptor; a new therapeutic use of a class of carboxamide compounds which are antagonists to a human 11CBy receptor; also to novel compounds within that class, and to methods for making the compounds.

International Patent Application Publication Number WO 01/21577 (Takeda Chemical Industries Ltd.) discloses certain bisaryl compounds as melanin concentrating hormone antagonists.

WO 98/00401 (Merck & Co. Inc.) discloses benzamide derivatives as fibrinogen receptor antagonist prodrugs.

European Patent EP 0 358 118 (Boehringer Mannheim GmbH) discloses certain bisaryl compounds as inhibitors of erythrocyte aggregation and useful in the treatment of cardiac and circulatory disease.

European Patent Application EP 0 968 999 (Mitsui Chemical Inc.) discloses certain anilide derivatives useful in the treatment of arrhythmia.

WO 99/01127 (SmithKline Beecham) discloses certain N-[(amino alkoxy)phenyl] benzamides that are active as CCR5 receptor ligands, including the compounds N-[4-[2-[bis(1-methylethyl)amino]ethoxy]-2-fluorophenyl]-[1,1′-biphenyl]-4-carboxamide and N-[4-[2-[bis(1-methylethyl)amino]-ethoxy]-phenyl]-[1,1′-biphenyl]4-carboxamide. Also WO 99/06146 (SmithKline Beecham) discloses certain substituted anilides that are antagonists of the CCR5 receptor, including the compounds: biphenyl-4-carboxylic acid [4-(2-dimethylamino-ethoxy)-phenyl]-amide, biphenyl-4-carboxylic acid [4(2-diisopropylamino-ethoxy)-phenyl]-amide, N-[4-(2-diisopropylamino-ethoxy)-phenyl]-4-phenoxy-benzamide, N-[4-(2-diethylamino-ethoxy)-phenyl]-4-phenoxy-benzamide, N-[4-(2-diisopropylamino-ethoxy)-phenyl]-3-phenoxy-benzamide, N-[4-(2-diethylamino-ethoxy)-phenyl]-3-phenoxy-benzamide, 4-cyclohexyl-N-[4-(2-diisopropylamino-ethoxy)-phenyl]-benzamide, 4-cyclohexyl-N-[4-(2-diethylamino-ethoxy)-phenyl]-benzamide, 4-benzyl-N-[4-(2-diisopropylamino-ethoxy)-phenyl]-benzamide, 4-benzyl-N-[4-(2-diethylamino-ethoxy)-phenyl]-benzamide, 4′-ethyl-biphenyl-4-carboxylic acid [4-(2-diisopropylamino-ethoxy)-phenyl]-amide, and 4′-ethyl-biphenyl-4-carboxylic acid [4-(2-diethylamino-ethoxy)-phenyl]-amide.

The present invention is based on the finding that a class of carboxamides overlapping with the above-mentioned benzamides and anilides, are, surprisingly, antagonists of a human 11CBy receptor disclosed in Nature, 400, 261-265 (1999).

Accordingly these compounds are believed to have a role in preventing, ameliorating or correcting dysfunctions or diseases, including, but not limited to, infections such as bacterial, fungal, protozoan and viral infections, particularly infection caused by HIV-1 or HIV-2; pain; cancers; diabetes; obesity; feeding and drinking abnormalities, such as anorexia and bulimia; asthma; Parkinson's disease; both acute and congestive heart failure; hypotension; hypertension; urinary retention; osteoporosis; angina pectoris; myocardial infarction; ulcers; allergies; benign prostatic hypertrophy; psychotic and neurological disorders, including anxiety, schizophrenia, manic depression, delirium, dementia or severe mental retardation; and dyskinesias, such as Huntington's disease or Gilles de la Tourette's syndrome, among others, hereinafter referred to as “the Disorders”.

According to the present invention there is provided a method of treating the Disorders which comprises administering to a mammal suffering from one or more of the Disorders an effective amount of a compound of formula (I), or a pharmaceutically acceptable salt or solvate thereof, in which:

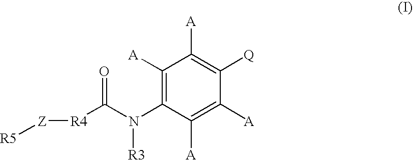

each A is independently hydrogen, a C₁₋₆ alkyl optionally substituted by hydroxyl, C₁₋₆ alkoxy, C₁₋₆ alkenyl or C₁₋₆ acyl group or a halogen atom or hydroxyl, CN or CF₃ group; R3 is hydrogen, methyl or ethyl.

Preferably R3 is methyl.

R4 is an optionally substituted aromatic carbocyclic or heterocyclic ring.

Z is an O or S atom, or an NH or CH₂ group, or a single bond, at the 3 or 4 position of R4 relative to the carbonyl group.

Preferably, Z is a bond.

More preferably, Z is a bond at the 4-position of R4 relative to the carbonyl group. R5 is an optionally substituted aromatic carbocyclic or heterocyclic ring, or an optionally substituted, saturated or unsaturated, carbocyclic or heterocyclic ring.

Preferably, R5 is a phenyl ring.

and Q is

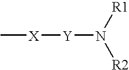

(a) where X is an O or S atom, preferably an O atom;

Y is a linear or branched C₂₋₄ alkylene group, preferably a C₃ alkylene group, optionally substituted by a hydroxyl group, or is a C₅₋₆ cycloalkylene group,

R1 and R2 are independently a linear or branched C₁₋₆ alkyl, preferably ethyl; phenyl C₁₋₆ alkyl group; or

(b) where X is an O or S atom;

Y is a linear or branched C₂₋₄ alkylene group, optionally substituted by a hydroxyl group,

R1 and R2 are linked to form a 5, 6 or 7-membered ring, preferably a 5-membered ring, optionally containing one or more further heteroatoms selected from O, S or N, where N or C ring atoms are optionally substituted by Ra, —CO-Ra, —CO—NH-Ra, or CO—O-Ra, where Ra is a linear or branched C₁₋₆ alkyl or aryl group; and the 5, 6 or 7-membered ring is optionally fused to an optionally substituted benzene ring, or a ring atom of the 5, 6 or 7-membered ring is optionally linked by a single bond or methylene group to Y; or

(c) where X is an O or S atom,

Y is a C₂₋₄ alkylene group, R1 is a C₂₋₄ alkylene group linked to Y to form a 5 or 6 membered ring and R2 is a linear or branched C₁₋₆ alkyl group; or

(d) where X is a N atom,

Y is a C₂₄ alkylene group, R1 is a C₂₋₄ alkylene group linked to X to form a 5 or 6 membered ring and R2 is a linear or branched C₁₋₆ alkyl group.

Alkyl groups, including alkyl groups that are part of alkoxy, acyl, etc groups, typically contain 1 to 6 carbon atoms, and may be linear or branched, such as methyl, ethyl, i-propyl and t-butyl, and optionally substituted by hydroxyl. Aryl groups are typically phenyl, but may include bicyclic groups such as naphthyl. Cycloalkyl groups typically contain from 3 to 7 carbon atoms. Heterocyclic groups may be monocylic 5 to 7 membered rings containing up to three hetero atoms, such as pyridyl or imidazole, or bicyclic, especially heterocyclic rings fused to benzene rings, such as benzoxazole or benzimidazole. Aryl, cycloalkyl and heterocyclic groups may be optionally subsituted by up to three substituents, which may suitably be selected from aryl, alkyl, alkoxy, halogen, hydroxy and cyano, or by linked substituents such as dioxymethylene.

Suitable aromatic rings for use as R4 include phenyl, pyridyl, thienyl, furanyl and pyrazolyl. Suitable optional substituents for R4 include halogen, CF₃, C₁₋₄ alkyl, C₁₋₄ alkoxy. R4 may have 2 or 3 substituents, but preferably has only 1 substituent in addition to Z, or more preferably is unsubstituted apart from Z. Particularly suitable substituents for R4 include chloro, fluoro, trifluoromethyl, methyl, methoxy.

R5 may be monocyclic, for example thienyl, furanyl, imidazolyl, oxadiazolyl, phenyl, pyridinyl, cyclohexyl, piperidinyl, piperazinyl, pyrazinyl, pyrimidinyl; or a fused bicyclic ring system, for example naphthyl, 3,4-dioxymethylene-phenyl, benzofuranyl, indolyl; or a bicyclic system in which a monocyclic ring has a cyclic substituent such as oxadiazolyl, benzyloxy. Suitable optional substituents for R5 include halogen, CF₃, CF₃O, CHF₂O, CN, amino, mono- or di-C₁₋₆ alkylamino, C₁₋₆ alkyl, C₁₋₆ alkoxy, C₁₋₆ acyl, C₁₋₆ alkyl-S—, C₁₋₆ alkyl-SO₂—, C₁₋₆ alkenyl, phenyl-C₁₋₆ alkyl, phenyl-C₁₋₆ alkoxy. R5 may have 2 or 3 substituents, but preferably has only 1 substituent, especially in the para position relative to Z. Particularly suitable substituents for R5 include chloro, fluoro, trifluoromethyl, cyano, amino, methyl, ethyl, t-butyl, methoxy, acetyl, formyl, methylthio, methanesulphonyl, vinyl, benzyl, benzyloxy, hydrogen.

As for the ring substituents A, all A substituents may be hydrogen, but it is advantageous that no more than 3 are hydrogen. Suitable A substituents include halogen, C₁₋₆ alkyl optionally substituted with hydroxy, C₁₋₆ alkoxy, C₁₋₆ acyl and C₁₋₆ alkenyl. Particularly suitable A substituents include C1-2alkoxy, C1-2alkyl, C1-2 acyl. Preferable substituents for A include chloro, fluoro, methyl, ethyl, hydroxyethyl, methoxy, formyl, acetyl, vinyl and allyl. More preferable substituents for A include methoxy. Suitably, the A substituent is adjacent to the group Q.

In the system Q, in configuration (a) particularly suitable substituents for R1 and R2 include methyl, ethyl, isopropyl, benzyl, phenethyl. Y may especially be —(CH₂)₂—, —(CH₂)₃—, —(CH₂)₄—, —CH₂—CH(CH₃)—CH₂—. When Y is substituted by hydroxy, it may be for example —CH₂—CH(OH)—CH₂—.

In configuration (b) of system Q, the ring formed by linking R1 and R2 may be pyrrolidinyl, piperidinyl, azepanyl, or imidazolyl. Fused rings include indolinyl, tetrahydroisoquinolinyl, tetrahydroquinolinyl and benzoazepinyl. When a second heteroatom is present, suitable rings include thiazinyl, oxazinyl and piperazinyl. A second N atom may be substituted, for example by phenyl, methyl, ethyl, isopropyl or acetyl. Y is typically —(CH₂)₂—. The ring may be linked back to Y to form a quinuclidinyl group.

In configuration (c) of system Q, the ring formed by linking R1 to Y may be a pyrrolidinyl or piperidinyl ring. The linkage to Y may be such as to create a ring linked by a single bond from a ring carbon atom directly to X or via a methylene or ethylene linking group. R2 is typically methyl so that the N atom of the ring is substituted by methyl.

In configuration (d) of system Q, the ring formed by linking R1 to N is suitably a 5 or 6-membered ring such as diazinyl or piperazinyl. Y is typically —(CH₂)₂—. R2 is typically methyl so that the second N atom (other than X) of the ring is substituted by methyl.

Within the scope of formula (I) is a class of compounds of general formula (II)

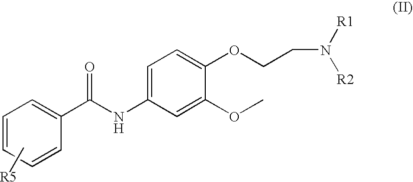

where A=H and OMe, R3=H, X=O, Y=CH₂CH₂, Z= a bond, R4=Ph, R5 is either meta or para substituted on R4, and R1, R2 and R5 are as defined for formula (I).

Also within the scope of formula (I) is a class of compounds of general formula (III)

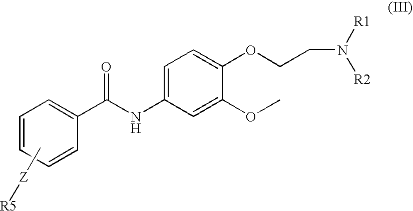

where A=H and OMe, R3=H, X=O, Y=CH₂—CH₂, Z=O, CH₂ or NH and is either meta or para substituted on R4, R4=Ph, R5 is Ph, and R1 and R2 are as defined for formula (I).

Also within the scope of formula (I) is a class of compounds of general formula (IV)

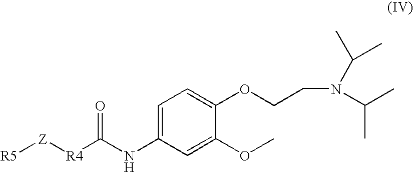

where A=H and OMe, R1=R2=iPr, R3=H, X=O, Y=CH₂—CH₂, and R4 and R5 are substituted phenyl or heterocycles as defined for formula (I)

Also within the scope of formula (I) is a class of compounds of general formula (V)

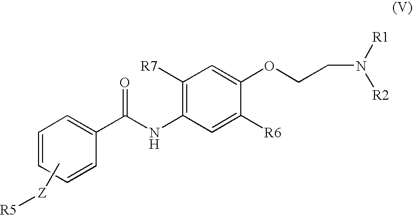

where R3=H, X=O, Y=CH₂—CH₂, Z=O, CH₂, NH or a bond, R4=Ph, R5 is Ph or cyclohexyl (Cy), Z is either meta or para substituted on R4, and A (R6, R7) and R1, R2 are as defined in formula (I).

Also within the scope of formula (I) is a class of compounds of general formula (VI)

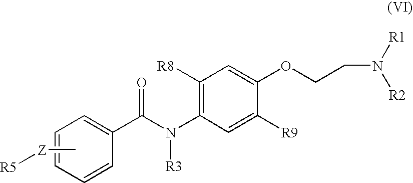

where X=O, Y=CH₂—CH₂, R4=phenyl, R5=phenyl or cyclohexyl (Cy), Z=O, CH₂ or a bond, and A R8, R9), R3 and R1, R2 are as defined in formula (I).

Also within the scope of formula (I) is a class of compounds of general formula (VII)

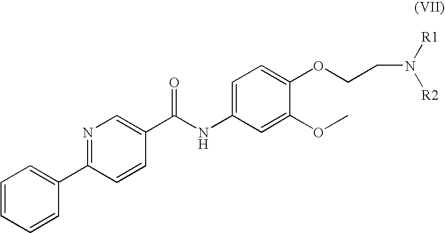

where A=H and OMe, X=O, R3=H, R4=3-pyridyl (with respect to the carbonyl group), R5=phenyl, Z=a para bond, and R1, R2 are as defined in formula (I).

Also within the scope of formula (I) is a class of compounds of general formula (VIII)

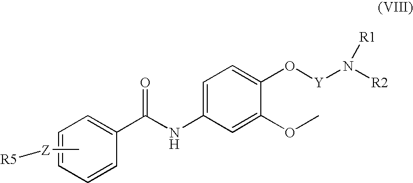

where A=H and OMe, R3=H, X=O, R4=phenyl, Z=O, C₂ or a bond, R5=Ph or cyclohexyl (Cy), Y is a chain of 3 or 4 carbon atoms optionally substituted by an hydroxyl group, and R1, R2 are as defined in formula (I).

Also within the scope of formula (I) is a class of compounds of general formula (IX)

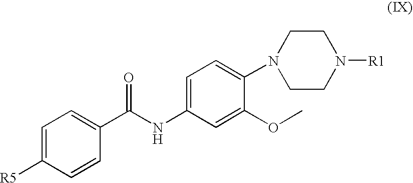

where A=H and OMe, R3=H, X=N, R4=phenyl, Z=a para substituted bond, R5=Ph or cyclohexyl (Cy), Y and R2 form a piperazinyl ring between X and N, and R1 is as defined in formula (I).

A preferred sub-class of compounds for use in the method of treatment of this invention are compounds of formula (I) in which R3 is methyl.

Within formula (I) is a novel group of compounds in which R3 is methyl or ethyl. The novel compounds, or a salt or solvate thereof, form a further aspect of this invention.

A particular group of novel compounds is a class of compounds of general formula (VI)

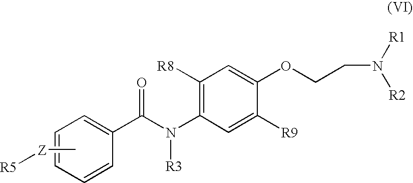

where R8 and R9 are as defined for A in formula (I), R1, R2 and R5 are as defined in formula (I), and R3 is methyl or ethyl.

Suitably R5 is phenyl or cyclohexyl optionally substituted by halogen, haloalkyl, alkyl or alkoxy; Z is O, CH₂ or a single bond; R8 and R9 are independently selected from hydrogen, halogen, alkyl and alkoxy; R1 and R2 are alkyl or linked together to form a ring; and R3 is ethyl or methyl.

Another aspect of this invention is a class of novel compounds, or a salt or solvate thereof, which are the compounds of formula (I) excluding the compounds:

N-[4-[2-[bis(1-methylethyl)amino]ethoxy]-2-fluorophenyl]-[1,1′-biphenyl]-4-carboxamide,

N-[4-[2-[bis(1-methylethyl)amino]ethoxy]phenyl]-[1,1′-biphenyl]-4-carboxamide, biphenyl-4-carboxylic acid [4-(2-diisopropylamino-ethoxy)-phenyl]-amide,

N-[4-(2-diisopropylamino-ethoxy)-phenyl]-4-phenoxy-benzamide,

N-[4-(2-diethylamino-ethoxy)-phenyl]-4-phenoxy-benzamide,

N-[4-(2-diisopropylamino-ethoxy)-phenyl]-3-phenoxy-benzamide,

N-[4-(2-diethylamino-ethoxy)-phenyl]-3-phenoxy-benzamide,

4-cyclohexyl-N-[4-(2-diisopropylamino-ethoxy)-phenyl]-benzamide,

4-cyclohexyl-N-[4-(2-diethylamino-ethoxy)-phenyl]-benzamide,

4-benzyl-N-[4-(2-diisopropylamino-ethoxy)-phenyl]-benzamide,

4-benzyl-N-[4-(2-diethylamino-ethoxy)-phenyl]-benzamide,

4′-ethyl-biphenyl-4-carboxylic acid [4-(2-diisopropylamino-ethoxy)-phenyl]-amide,

and 4′-ethyl-biphenyl-4-carboxylic acid [4-(2-diethylamino-ethoxy)-phenyl]-amide.

A further aspect of this invention is those compounds of the Examples herein which are novel.

The compounds of formulae (I) to (IX), or their salts or solvates, are preferably in pharmaceutically acceptable or substantially pure form. By pharmaceutically acceptable form is meant, inter alia, of a pharmaceutically acceptable level of purity excluding normal pharmaceutical additives such as diluents and carriers, and including no material considered toxic at normal dosage levels.

Suitable salts and solvates include pharmaceutically acceptable salts and pharmaceutically acceptable solvates.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-β-phenethylamine, dehydroabietylamine, N,N′-bisdehydroabietylamine, glucamine, N-methylglucamine or bases of the pyridine type such as pyridine, collidine, quinine or quinoline.

Suitable pharmaceutically acceptable salts also includes pharmaceutically acceptable acid addition salts, such as those provided by pharmaceutically acceptable inorganic acids or organic acids.

Suitable pharmaceutically acceptable acid addition salts provided by pharmaceutically acceptable inorganic acids includes the sulphate, nitrate, phosphate, borate, hydrochloride and hydrobromide and hydroiodide.

Suitable pharmaceutically acceptable acid addition salts provided by pharmaceutically acceptable organic acids includes the acetate, tartrate, maleate, fumarate, malonate, citrate, succinate, lactate, oxalate, benzoate, ascorbate, methanesulphonate, α-keto glutarate and α-glycerophosphate.

Suitable pharmaceutically acceptable solvates include hydrates.

A substantially pure form will generally contain at least 50% (excluding normal pharmaceutical additives), preferably 75%, more preferably 90% and still more preferably 95% of the compound of formula (I) to (IX) or its salt or solvate.

One preferred pharmaceutically acceptable form is the crystalline form, including such form in a pharmaceutical composition. In the case of salts and solvates the additional ionic and solvent moieties must also be non-toxic.

Examples of pharmaceutically acceptable salts of a compound of formula (I) to (IX) include the acid addition salts with the conventional pharmaceutical acids, for example, maleic, hydrochloric, hydrobromic, phosphoric, acetic, fumaric, salicylic, citric, lactic, mandelic, tartaric, succinic, benzoic, ascorbic and methanesulphonic.

The compounds of formula (I) to (IX) may exist in more than one stereoisomeric form, and the invention extends to all such forms as well as to their mixtures thereof, including racemates.

The compounds of formula (I) to (IX), or salts or solvates thereof, may be prepared by the methods illustrated in the following general reaction schemes, or by modification thereof, using readily available starting materials, reagents and conventional synthetic procedures. If a particular enantiomer of a compound of the present invention is desired, it may be synthesised starting from the desired enantiomer of the starting material and performing reactions not involving racemization processes or it may be prepared by chiral synthesis, or by derivatisation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxy, diastereomeric salts are formed with an appropriate optically active acid or base, followed by resolution of diastereomeric salts by fractional crystallization and subsequent recovery of the pure enantiomers.

Compounds of formula (I) to (IX) may prepared by condensing suitably substituted aryl or heteroarylcarboxylic acids and suitably substituted anilines, which are commercially available or synthesized by methods known to the art from commercially available starting materials, using methods known to the art. For example, suitably substituted aryl or heteroarylcarboxylic acids are treated with an activating reagent, such as thionyl chloride, at a suitable temperature, such as at reflux, to afford aryl or heteroarylcarbonyl chlorides, and the aryl- or heteroarylcarbonyl chlorides are condensed with suitably substituted anilines in the presence of a suitable base, such as diisopropylethylamine, in a suitable solvent, such as dichloromethane, to give compounds of formula (I).

In particular, the preparation of certain carboxamides of formula (I) in which R3 is H is disclosed in WO 99/01127 and WO 99/06146 mentioned above, and analogous methods of preparation may be used in the present invention. Many additional methods for converting a carboxylic acid to an amide are known, and can be found in standard reference books such as “Compendium of Organic Synthetic Methods”, Vol. I-VI (published by Wiley-Interscience).

For example the compounds of formula (I) may be prepared by reacting a compound of formula (X)

R5-Z-R4-COL (X)

where L is a leaving group such as halogen, especially chlorine or bromine with a compound of formula (XI)

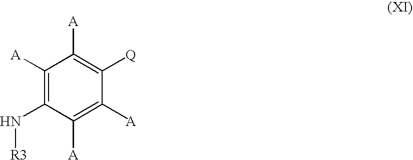

where A, Z, R3, R4, R5 and Q are as defined for formula (I).

In this process, groups convertible to R1, R2, R3, R4 and R5 may be present during the coupling, and converted to R1, R2, R3, R4 and R5 after coupling. Also it may be convenient to convert one R1, R2, R3, R4 and R5 to another R1, R2, R3, R4 and R5 group after coupling. In particular, ring formation between the groups R1, X, Y, R2 or the addition of suitable cyclic groups embodying R1, X, Y, R2, may be performed after coupling.

Accordingly, there is provided a process for the preparation of a compound of formula (I), or a salt or solvate thereof, wherein R3 is methyl or ethyl which process comprises the reaction of a compound of formula (X) as hereinbefore defined with a compound of formula (XI) wherein A and Q are as hereinbefore defined and R3 is methyl or ethyl.

There therefore also provided a process for the preparation of a compound of formula (I), or a salt or solvate thereof, with the proviso that the following compounds are excluded;

N-[4-[2-[bis(1-methylethyl)amino]ethoxy]-2-fluorophenyl]-[1,1′-biphenyl]4-carboxamide,

N-[4-[2-[bis(1-methylethyl)amino]ethoxy]phenyl]-[1,1′-biphenyl]4-carboxamide, biphenyl-4-carboxylic acid [4-(2-diisopropylamino-ethoxy)-phenyl]-amide,

N-[4-(2-diisopropylamino-ethoxy)-phenyl]A4-phenoxy-benzamide,

N-[4-(2-diethylamino-ethoxy)-phenyl]-4-phenoxy-benzamide,

N-[4-(2-diisopropylamino-ethoxy)-phenyl]-3-phenoxy-benzamide,

N-[4-(2-diethylamino-ethoxy)-phenyl]-3-phenoxy-benzamide,

4-cyclohexyl-N-[4-(2-diisopropylamino-ethoxy)-phenyl]-benzamide,

4-cyclohexyl-N-[4-(2-diethylamino-ethoxy)-phenyl]-benzamide,

4-benzyl-N-[4-(2-diisopropylamino-ethoxy)-phenyl]-benzamide,

4-benzyl-N-[4-(2-diethylamino-ethoxy)-phenyl]-benzamide,

4′-ethyl-biphenyl-4-carboxylic acid [4-(2-diisopropylamino-ethoxy)-phenyl]-amide,

and 4′-ethyl-biphenyl-4-carboxylic acid [4-(2-diethylamino-ethoxy)-phenyl]-amide.

which process comprises the reaction of a compound of formula (X) as hereinbefore defined with a compound of formula (XI) as hereinbefore defined.

The compounds of formula (XI) may be prepared in a number of ways, for example when X is O or S coupling an appropriately substituted nitrobenzene compound with a dialkyaminoalcohol or thiol, and converting the NO₂ group to NH₂ by hydrogenation in the presence of palladium catalyst (or with iron/ammonium chloride) before coupling with an acid chloride, for example as illustrated below:

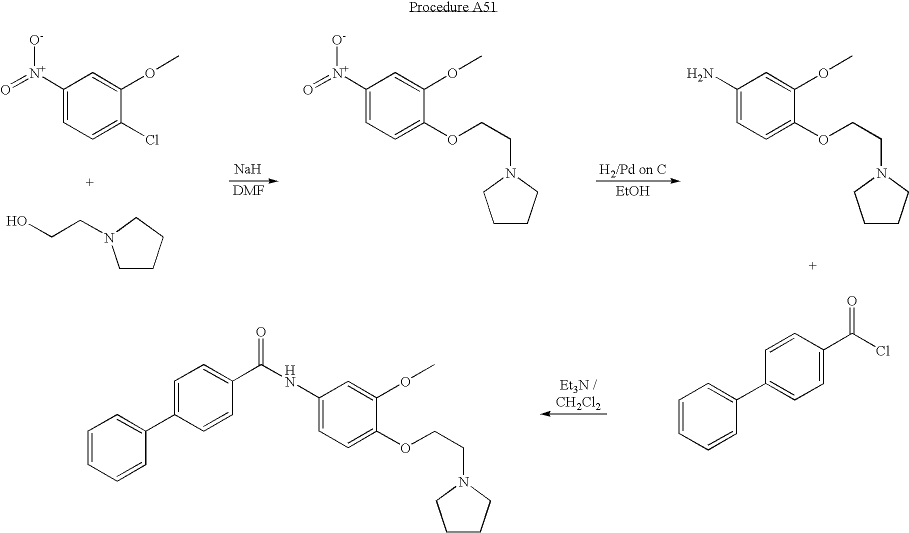

Acid chlorides of formula (X) may be prepared from the corresponding acids which are commercially available or described in the literature or may be prepared by methods analogous to those of the literature.

Alternatively the acids of formula (X) may be prepared by combining moieties containing respectively R5 and R4 via Z.

This may also be achieved conveniently by first coupling a compound of R4-CO-L with the compound of formula (XI) followed by reaction with a compound R5-Z-L (or L-R4-CO-L with R5-Z). For example an amine of formula (XI) may be reacted with an appropriately substituted bromobenzoyl chloride which may be then reacted with, for example, an appropriately substituted phenyl moiety with a leaving group, or a cyclic amine, as in the following scheme:

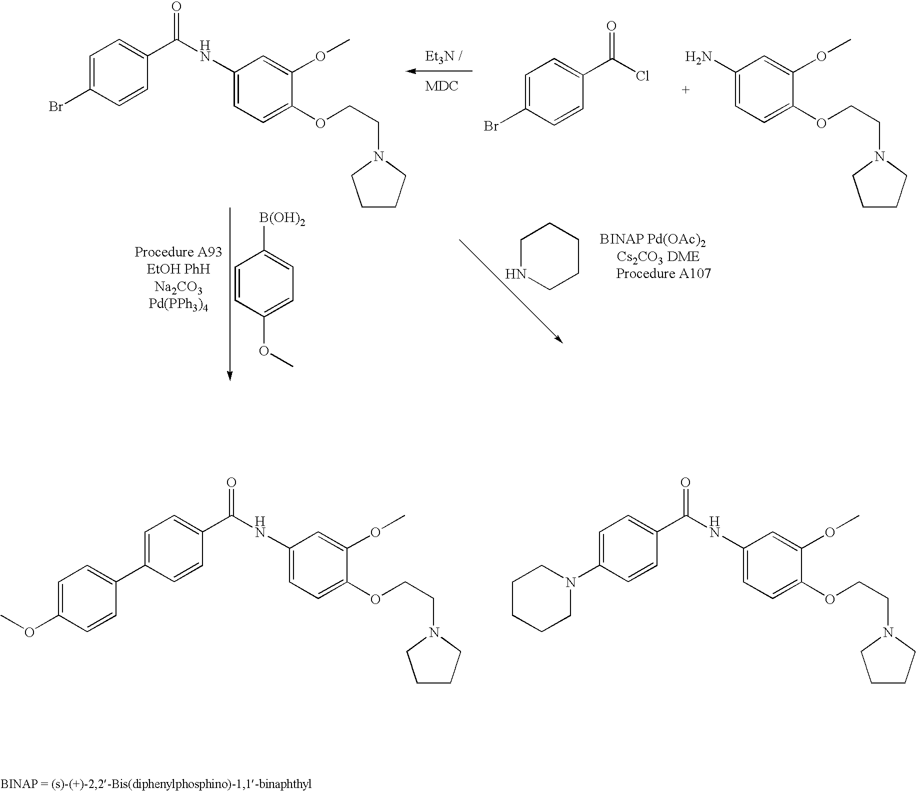

Similar reactions building up the structure of formula (I) may be carried out starting with the compound of formula (X) and adding the equivalent of formula (XI) in sections, as in the scheme below where an N-protecting group on Q, here a piperazine ring, may be removed after coupling the components of formula (I) and replacement by a desired substituent:

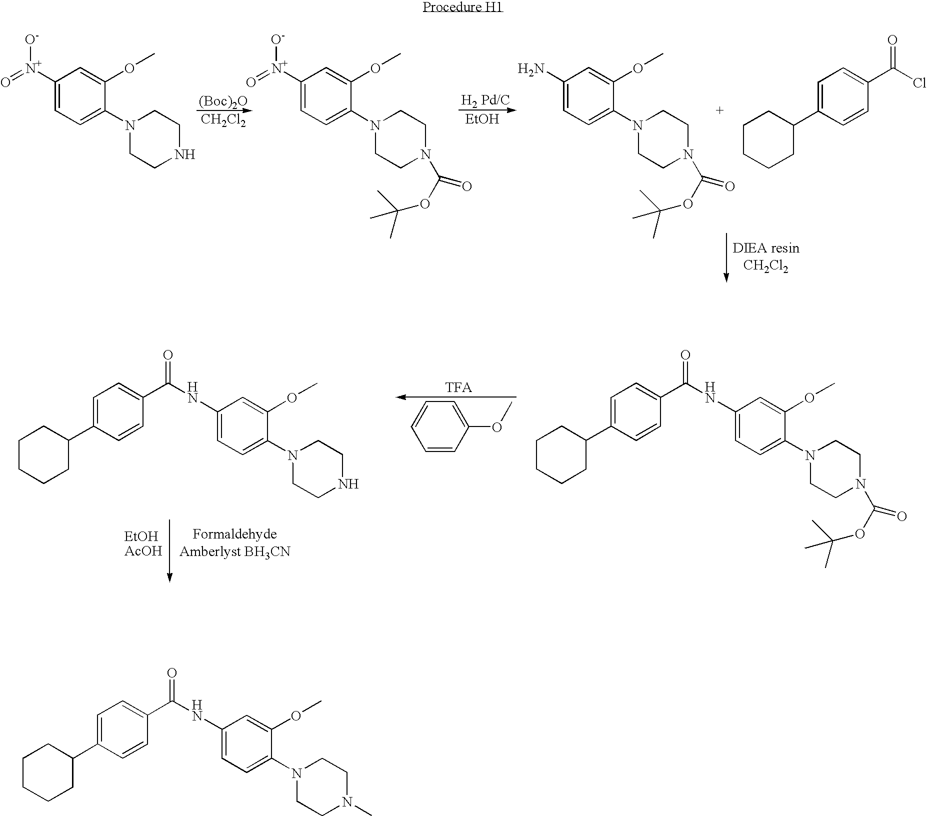

In an alternative strategy for building up the compounds of formula (XI) before coupling, so as to introduce a hydroxy group in Y, an appropriately substituted nitrophenol is linked to an epoxy compound which is then reacted with an amine forming a group Q which is —O—Y(OH)-NR1R2, before coupling with R5-Z-R4-CO-L, as illustrated by:

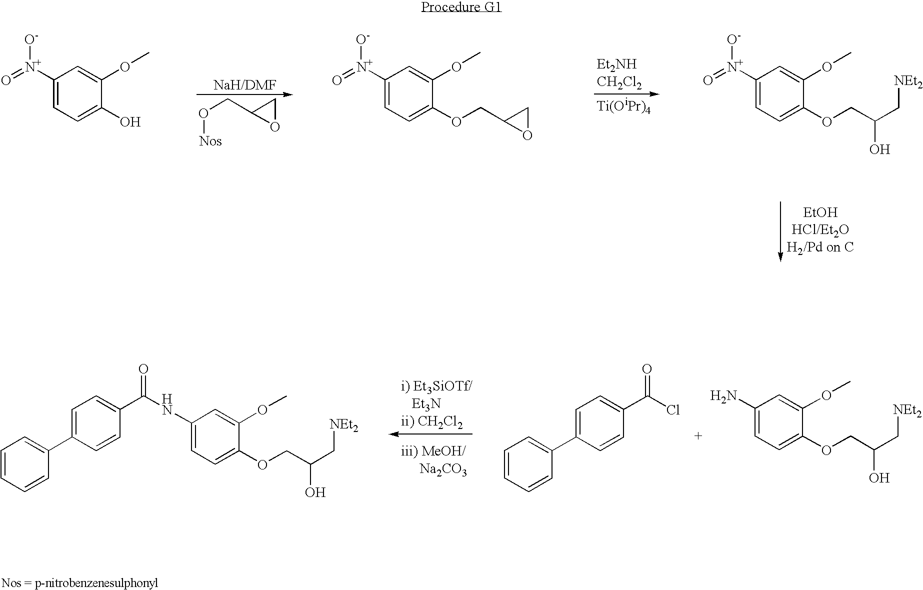

Novel compounds of formula (I) where the amide nitrogen is alkylated (R3 is methyl or ethyl) may be prepared by alkylating an anilide of formula M(I) before coupling with an acid chloride of formula (X), for example, by utilising the following reductive amination procedure:

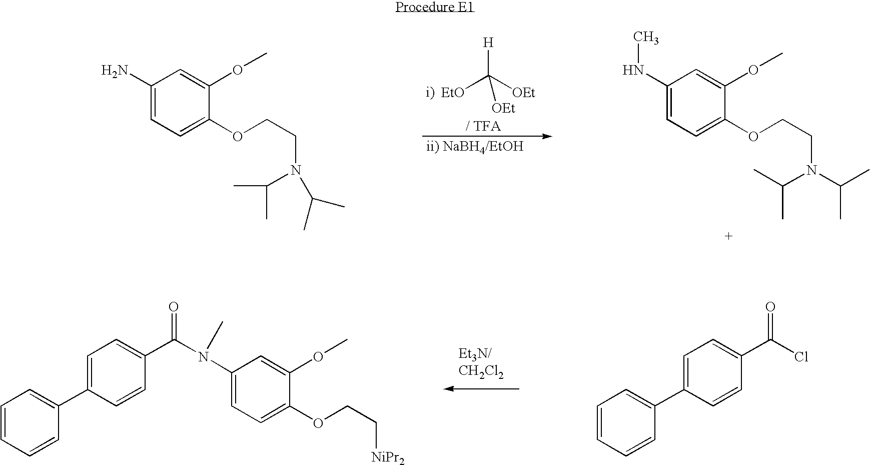

The compounds of formula (I) may be converted into their pharmaceutically acceptable salts by reaction with the appropriate organic or mineral acids.

Solvates of the compounds of formula (I) may be formed by crystallization or recrystallization from the appropriate solvent. For example, hydrates may be formed by crystallization or recrystallization from aqueous solutions, or solutions in organic solvents containing water.

Also salts or solvates of the compounds of formula (I) which are not pharmaceutically acceptable may be useful as intermediates in the production of pharmaceutically acceptable salts or solvates. Accordingly such salts or solvates also form part of this invention.

The above-listed compounds and pharmaceutically acceptable salts thereof, especially the hydrochloride, and pharmaceutically acceptable solvates, especially hydrates, form a preferred aspect of the present invention.

By virtue of the activity of these compounds as antagonists of a human 11CBy receptor, the compounds of formula (I) are believed to have a role in preventing, ameliorating or correcting dysfunctions of diseases, including, but not limited to, “the Disorders” previously mentioned.

It is also considered that the treatment of certain of the Disorders mentioned above by an antagonist to the human 11CBy receptor are novel. Accordingly, the invention also provides a method for the treatment of diabetes, major depression, manic depression, anxiety, schizophrenia and sleep disorders, in human or non-human mammals which method comprises the administration of a therapeutically effective amount of an antagonist to the human 11CBy receptor. In particular the the invention provides a method for the treatment of diabetes in human or non-human mammals which method comprises the administration of a therapeutically effective amount of an antagonist to the human 11CBy receptor. In particular the invention provides a method for the treatment of major depression, in human or non-human mammals which method comprises the administration of a therapeutically effective amount of an antagonist to the human 11CBy receptor. In particular the invention provides a method for the treatment of manic depression, in human or non-human mammals which method comprises the administration of a therapeutically effective amount of an antagonist to the human 11CBy receptor. In particular the the invention provides a method for the treatment of anxiety in human or non-human mammals which method comprises the administration of a therapeutically effective amount of an antagonist to the human 11CBy receptor. In particular the the invention provides a method for the treatment of schizophrenia in human or non-human mammals which method comprises the administration of a therapeutically effective amount of an antagonist to the human 11CBy receptor.

In particular the the invention provides a method for the treatment of sleep disorders, in human or non-human mammals which method comprises the administration of a therapeutically effective amount of an antagonist to the human 11CBy receptor.

The administration of such compounds to a mammal may be by way of oral (including sub-lingual), parenteral, nasal, rectal or transdermal administration.

An amount effective to treat the Disorders hereinbefore described depends on the usual factors such as the nature and severity of the disorders being treated and the weight of the mammal. However, a unit dose will normally contain 1 to 1000 mg, suitably 1 to 500 mg, for example an amount in the range of from 2 to 400 mg such as 2, 5, 10, 20, 30, 40, 50, 100, 200, 300 and 400 mg of the active compound. Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

It is greatly preferred that compounds of formula (I) are administered in the form of a unit-dose composition, such as a unit dose oral (including sub-lingual), nasal, rectal, topical or parenteral (especially intravenous) composition.

Such compositions are prepared by admixture and are suitably adapted for oral or parenteral administration, and as such may be in the form of tablets, capsules, oral liquid preparations, powders, granules, lozenges, reconstitutable powders, injectable and infusable solutions or suspensions or suppositories. Orally administrable compositions are preferred, in particular shaped oral compositions, since they are more convenient for general use.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

These solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats; emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

Oral formulations also include conventional sustained release formulations, such as tablets or granules having an enteric coating.

For parenteral administration, fluid unit dose forms are prepared containing the compound and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and is sterilised by exposure to ethylene oxide before suspending in the sterile vehicle. Advantageously, a surfactant or wetting agent is included in the composition to facilitate uniform distribution of the compound of the invention.

As is common practice, the compositions will usually be accompanied by written or printed directions for use in the medical treatment concerned.

Compounds of the present invention may be employed alone or in conjunction with other compounds, such as therapeutic compounds.

No adverse toxicological effects are expected for the compounds of the invention, when administered in accordance with the invention.

Accordingly, in a further aspect, the present invention provides a pharmaceutical composition for use in the treatment and/or prophylaxis of one or more of the Disorders which comprises a compound of this invention, or a pharmaceutically acceptable salt or solvate thereof, and a pharmaceutically acceptable carrier.

The present invention also provides a method of treatment and/or prophylaxis of one or more of the Disorders comprising administering to the sufferer in need thereof an effective or prophylactic amount of a compound of this invention, or a pharmaceutically acceptable salt or solvate thereof.

In a further aspect the invention provides the use of a compound of this invention, or a pharmaceutically acceptable salt or solvate thereof, for the manufacture of a medicament for the treatment and/or prophylaxis of one or more of the Disorders.

In a still further aspect the invention provides the use of a novel compound of this invention, or a pharmaceutically acceptable salt or solvate, thereof as a therapeutic agent, in particular for the treatment and/or prophylaxis of one or more of the Disorders.

Compounds for use in this invention and their preparation are illustrated in the following Examples and Tables.

These Examples illustrate general procedures and sources of chemicals utilised to prepare compounds whose structures are shown in the Tables of data which follow the Examples. In the case of Examples prepared as members of a coupled array, the synthetic origin of all starting componants of the array are shown in the Examples. Rather than detailing the experimental procedure for each case, the method by which individual members of the array were prepared is indicated in a Table by reference to a related Example. Mass spectral characterisation of all Examples is provided in the tables of data. Additional characterisation is provided for selected representative Examples with full experimental procedures.

### EXAMPLE A1

### WO-00/06146

Utilising the procedure of Example A7 with 4-biphenylcarboxylic acid [Aldrich] in place of 2′-methyl-4-biphenylcarboxylic acid.

### EXAMPLE A2

Correspondingly Example A7 with 4-(5-methyl-[1,2,4]oxadiazol-3-yl)-benzoic acid [J. Org. Chem. 50; 8; 1985; 1182].

### EXAMPLE A3

Correspondingly Example A7 with 4-pyrazol-1-yl-benzoic acid [Can. J. Chem.; 41; 1963; 1540].

### EXAMPLE A4

Correspondingly Example A7 with 3-biphenylcarboxylic [Med. Chem. Res.; 6; 2; 1996].

### EXAMPLE A5

Correspondingly Example A7 with 4-(2-pyridyl)-benzoic acid [J. Chem. Soc.; 1940; 355, 356].

### EXAMPLE A6

Correspondingly Example A7 with 3′-acetyl-biphenyl-4-carboxylic acid [Patent WO-9743262].

### EXAMPLE A7

2-methylphenyl-4-phenylcarboxylic acid [3-methoxy-4-(2-bis-(2-methylethyl)amino)-ethoxy)-phenyl amide.

To a solution of the acid (2′-methyl-biphenyl-4-carboxylic acid) [Patent WO-9901127] (55 mg, 0.26 mmol) in dimethylformamide were added (1-(3-dimethylaminopropyl)-3-ethyl carbodiimide hydrochloride [Aldrich] (50 mg, 0.26 mmol) and 1-hydroxy-7-azabenzotriazole [Aldrich] (35 mg, 0.26 mmol) followed by diisopropylethylamine (0.04 ml, 0.25 mmol) and the aniline (4-(2-diisopropylamino-ethoxy)-3-methoxy-phenylamine) (69 mg, 0.22 mmol), [prepared using the method used to form 3-methoxy-4-(2-pyrrolidin-1-yl-ethoxy)-phenylamine in Example A51 but with 2-diisopropylamino-ethanol in place of 1-(2-hydroxyethyl)-pyrrolidine]. The reaction mixture was stirred at room temperature for 16 hours. The solvent was evaporated, and the residue re-dissolved in dichloromethane (10 ml), filtered through an SAX [Varian] column (2 g), and the filtrate was then stirred with PS-isocyanate resin [Argonaut Technologies] (100 mg, 0.38 mmol) for 16 hours. The mixture was filtered, evaporated, and the residue purified by flash chromatography on silica gel using dichloromethane—aq. ammonia—methanol as eluent, to afford the title compound as an oil.

¹H NMR (CDCl₃): δ 1.04 (12H, d), 2.28 (3H, s), 2.90 (2H, t), 3.05 (2H, m), 3.91 (3H, s), 3.95 (2H, t), 6.88 (1H, d), 7.03 (1H, dd), 7.27-7.32 (4H, m), 7.44, (2H, d), 7.53 (1H, d), 7.94 (2H, d) and 8.01 (1H, bs); MS (AP+ve): m/z 461 [M+H]⁺.

### EXAMPLE A8

Utilising the procedure of Example A7 with cyclohexyl-4-benzoic acid [Aldrich], in place of (2′-methyl-biphenyl-4-carboxylic acid).

### EXAMPLE A9

Correspondingly Example A7 with 4-(2-thienyl)-benzoic acid [J. Chem. Soc. Perkin Trans.1; 17; 1992; 2203].

### EXAMPLE A10

Correspondingly Example A7 with 4-(1-methyl-1H-pyrazol-4-yl)-benzoic acid [Patent:WO-9906409].

### EXAMPLE A11

Correspondingly Example A7 with 4′-(5-methyl-[1,2,4]oxadiazol-3-yl)-biphenyl-4-carboxylic acid [Patent:WO-9743262].

### EXAMPLE A12

Correspondingly Example A7 with 4-benzyl-carboxylic acid [Apin].

### EXAMPLE A13

Correspondingly Example A7 with 3′-cyano-biphenyl-3-carboxylic acid [J. Chem. Soc. Perkin Trans. 2; 1; 1984; 35-38].

### EXAMPLE A14

Correspondingly Example A7 with 3′-methanesulfonyl-biphenyl-4-carboxylic acid [Izv. Sib. Otd. Akad. Nauk SSSR Ser. Khim. Nauk; 11; 1966; 62].

### EXAMPLE A15

Correspondingly Example A7 with 3-thiophen-2-yl-benzoic acid [Tetrahedron Lett.; 39; 24;1998;4175].

### EXAMPLE A16

Correspondingly Example A7 with 3-thiophen-3-yl-benzoic acid [J. Chem. Soc. B; 1970; 1595].

### EXAMPLE A17

Correspondingly Example A7 with 4-acetyl-4-biphenylcarboxylic acid [Aldrich].

### EXAMPLE A18

Correspondingly Example A7 with 4′-cyano-3′-methylbiphenyl-4-carboxylic acid [WO-9850358].

### EXAMPLE A19

Correspondingly Example A7 with 4′-(5-methyl-[1,3,4]oxadiazol-2-yl)-biphenyl-4-carboxylic acid [Patent:WO-9743262].

### EXAMPLE A20

Correspondingly Example A7 with 4-thiophen-3-yl-benzoic acid [J. Chem. Soc. B; 1970; 1595].

### EXAMPLE A21

Correspondingly Example A7 with 4-pyrazin-2-yl-benzoic acid [Patent WO-9854164].

### EXAMPLE A22

Utilising the procedures of Example A93 with 2-methoxyphenylboronic acid [Aldrich] in place of 4-methylphenylboronic acid, and Example A51 with 2-(diisopropylamino)-ethanol in place of 1-(2-hydroxyethyl)-pyrrolidine.

### EXAMPLE A23

Utilising the procedure of Example A22 with 4-trifluoromethylphenylboronic acid [Aldrich], in place of 2-methoxyphenylboronic acid [Aldrich]

### EXAMPLE A24

Correspondingly Example A23 with 3-aminophenylboronic acid [Aldrich].

### EXAMPLE A25

Correspondingly Example A23 with 4-benzyloxyphenylboronic acid Lancaster].

### EXAMPLE A26

Correspondingly Example A23 with 2-naphthylboronic acid [Lancaster].

### EXAMPLE A27

Correspondingly Example A23 with 3-naphthylboronic acid [Lancaster].

### EXAMPLE A28

Correspondingly Example A23 with 4-methylphenylboronic acid [Lancaster].

### EXAMPLE A29

Correspondingly Example A23 with 4-methylthiophenylboronic acid [Lancaster].

### EXAMPLE A30

Correspondingly Example A23 with 3-trifluoromethylphenylboronic acid [Lancaster].

### EXAMPLE A31

Correspondingly Example A23 with 4-carbonylphenylboronic acid [Aldrich].

### EXAMPLE A32

Correspondingly Example A23 with 3,4-(methylenedioxy)phenylboronic acid [Aldrich].

### EXAMPLE A33

Correspondingly Example A23 with 4-vinylphenylboronic acid [Aldrich].

### EXAMPLE A34

Correspondingly Example A23 with 3-methoxyphenylboronic acid [Lancaster].

### EXAMPLE A35

Utilising the procedure of Example A51 with 1-(2-hydroxyethyl)morpholine [Aldrich] in place of 1-(2-hydroxyethyl)pyrrolidine.

### EXAMPLE A36

Utilising the procedure of Example A35 with 4-cyclohexylbenzoic acid [Aldrich]. in place of 4-biphenylcarboxylic acid.

### EXAMPLE A37

Utilising the procedure of Example A51 with 2-dimethylaminoethanol [Aldrich], in place of 1-(2-hydroxyethyl)pyrrolidine.

### EXAMPLE A39

Correspondingly Example A51 with (R)-(+)-1-methyl-2-pyrrolidinemethanol (Patent WO-9932480).

### EXAMPLE A41

Correspondingly Example A51 with 3-hydroxy-1-methylpiperidine [Aldrich].

### EXAMPLE A43

Correspondingly Example A51 with 2-dimethylamino-1-propanol [ICN-RF].

### EXAMPLE A45

Correspondingly Example A51 with 2-(diethylamino)-ethanol [Aldrich].

### EXAMPLE A47

Correspondingly Example A51 with (S)-(−)-1-methyl-2-pyrrolidinemethanol [Aldrich].

EXAMPLE A49

Correspondingly Example A51 with N-benzyl-N-methylethanolamine [Aldrich].

EXAMPLE A51

Biphenyl-4-carboxylic acid [3-methoxy-4-(2-pyrrolidin-1-yl-ethoxy)-phenyl amide. To a solution of the hydroxy amine, (1-(2-hydroxyethyl)-pyrrolidine) [Aldrich], (1.87 ml, 16 mmol) in dimethylformamide was added portionwise sodium hydride [60% dispersion in oil, (544 mg, 16 mmol). After stirring at room temperature for 10 minutes a solution of the halonitrobenzene, (1-chloro-2-methoxy-4-nitro-benzene) [Avocado] (3 g, 16 mmol) in dimethylformamide (10 ml) was added dropwise. The reaction mixture was left stirring at room temperature for 16 hrs then concentrated. The residue was dissolved in ethyl acetate (2001 ml) and washed with water (3×50 ml). The organic phase was dried with magnesium sulphate, evaporated and the residue purified by flash chromatography on silica gel using dichloromethane—aq. ammonia—methanol as eluent to afford 1-[2-(2-methoxy-4-nitro-phenoxy)-ethyl]-pyrrolidine as a brown oil.

¹H NMR (CDCl₃): δ 1.82 (4H, m), 2.65 (4H, m), 3.01 (2H, t), 3.94 (3H, s), 4.24 (2H, t), 6.92 (1H, d), 7.74 (1H, d), and 7.89 (1H, dd); MS (AP+ve): m/z 267 [M+H]⁺.

To a solution of the amine, 1-[2-(2-methoxy-4-nitro-phenoxy)-ethyl]-pyrrolidine (2.3 g, 8.6 mmol) in ethanol (100 ml) was added 10% Pd/C (50 mg). The mixture was stirred at room temperature under an atmosphere of hydrogen at atmospheric pressure for 16 h, then filtered through celite and the filtrate concentrated to give the corresponding aniline; 3-methoxy-4-(2-pyrrolidin-1-yl-ethoxy)-phenylamine, as a brown solid.

¹H NMR (CDCl₃): δ 1.80 (4H, m), 2.62 (4H, m), 2.89 (2H, t), 3.80 (3H, s), 4.06 (2 h, t), 6.20 (1H, dd), 6.29 (1H, d) and 6.75 (1H, d); MS (AP+ve): m/z 237 [M+H]⁺.

To the carboxylic acid, (4-biphenyl carboxylic acid) [Aldrich] (47.5 mg, 0.24 mmol) suspended in dichloromethane (1 ml) was added oxalyl chloride [Aldrich] (0.06 ml, 0.72 mmol) followed by one drop of dimethylformamide. The reaction mixture was stirred at room temperature for 1 hour, concentrated, then co-evaporated three times with dichloromethane to give 4-phenylbenzoyl chloride. This was dissolved in dichloromethane (1 ml) and added to a solution containing the amine, (3-methoxy-4-(2-pyrrolidin-1-yl-ethoxy)-phenylamine), (47 mg, 0.2 mmol), triethylamine (0.14 ml, 1 mmol) and dichloromethane (1 ml). The reaction mixture was stirred for 16 hours at room temperature, concentrated, re-dissolved in dichloromethane (10 ml), filtered through an SAX column [Varian] (2 g) and stirred with PS-isocyanate resin [Argonaut Technologies] (100 mg, 0.38 mmol) for 16 hours. The mixture was filtered, evaporated then purified by flash chromatography on silica gel using dichloromethane—aq. ammonia—methanol as eluent to afford the title compound as an oil.

¹H NMR (CDCl₃): δ 1.88 (4H, m), 2.90 (4H, m), 3.08 (2H, t), 3.84 (3H, s), 4.21 (2H, t), 6.83 (1H, d), 7.03 (1H, dd), 7.27-7.70 (8H, m) and 8.01 (2H, d); MS (AP+ve): m/z 417 [M+H]⁺.

### EXAMPLE A54

Utilising the procedure of Example A51 with 1-dimethylamino-2-propanol [Aldrich] in place of 1-(2-hydroxyethyl)-pyrrolidine.

### EXAMPLE A56

Correspondingly Example A51 with 1-(2-hydroxyethyl)-piperidine [Aldrich].

### EXAMPLE A58

Correspondingly Example A51 with 2-(hexamethyleneamino)-ethanol [Lancaster].

### EXAMPLE A60

Utilising the procedures of Example A93 with 3-aminophenylboronic acid in place of 2-methoxyphenylboronic acid and Example 51 with 2-dimethylaminoethanol in place of 1-(2-hydroxyethyl)pyrrolidine.

### EXAMPLE A63

Utilising the procedure of Example A60 with 4-carboxyphenylboronic acid [Aldrich] in place of 3-aminophenylboronic acid.

### EXAMPLE A70

Correspondingly Example A63 with (3,4-methylenedioxyphenyl)boronic acid [Aldrich].

### EXAMPLE A72

Utilising the procedure of Example 51 with N-(2-phenyl)-ethyl-N-methyl-ethanolamine [J. Org. Chem. 1985, 50(22), 4359] in place of 1-(2-hydroxyethyl)-pyrrolidine.

### EXAMPLE A74

Correspondingly Example 51 with 2-dimethylaminocyclohexanol [J. Chem. Soc. C (1969), (2), 248-52].

### EXAMPLE A76

Correspondingly Example 51 with 2-(1,2,4,5-tetrahydro-benzo[d]azepin-3-yl)-ethanol [U.S. Pat. No. 394,682]

### EXAMPLE A78

Correspondingly Example 51 with 2-(3,4-dihydro-1H-isoquinolin-2-yl)-ethanol [Patent WO-9719926].

### EXAMPLE A80

Correspondingly Example 51 with 2-(4-phenyl-piperazin-1-yl)-ethanol [J. Med. Chem. 1994, 37(13), 1964].

### EXAMPLE A82

Correspondingly Example 51 with 1-methyl-3-pyrrolidinol [Aldrich].

### EXAMPLE A84

Utilising the procedures of Example A93 with 4-methoxy-phenylboronic acid [Aldrich] in place of 2-methoxyphenylboronic acid and Example A51 with 2-diethylaminoethanol in place of 1-(2-hydroxyethyl)pyrrolidine.

### EXAMPLE A88

Utilising the procedures of Example A84 with 4-methoxy-3-pyridylboronic acid [Patent WO-9924440] in place of 4-methoxy-phenylboronic acid.

### EXAMPLE A89

Correspondingly Example A88 with 2-methoxy-3-pyridylboronic acid [Patent WO-9910331].

### EXAMPLE A90

Correspondingly Example A88 with benzo-[b]-furan-2-boronic acid [Aldrich].

### EXAMPLE A91

Correspondingly Example A88 with thiophene-3-boronic acid [Aldrich].

### EXAMPLE A92

Correspondingly Example A88 with indole-5-boronic acid [Frontier].

### EXAMPLE A93

4′-Methyl-biphenyl-4-carboxylic acid [3-methoxy-4-(2-pyrrolidin-1-yl-ethoxy)-phenyl]-amide

A mixture of 3-methoxy-4-(2-pyrrolidin-1-yl-ethoxy)-phenylamine (Example A51] (4.7 mM 1.1 g) and triethylamine (14 mmol) was treated with 4-bromobenzoyl chloride [Aldrich] in dichloromethane (20 ml) and kept at room temperature for 16 hours. The solvent was evaporated and the crude product purified by chromatography on silica gel using dichloromethane—methanol—aq. ammonia to afford 4-bromo-N-[3-methoxy-4(2-pyrrolidin-1-yl-ethoxy)-phenyl]-benzamide as a white solid in 72% yield.

¹H NMR (DMSO-d₆): δ 7.91 (2H, dd), 7.73 (2H, dd), 7.50 (1H, d), 7.30 (1H, dd), 6.94 (1H, d), 4.02 (2H, t), 3.76 (3H, s), 2.77 (2H, t), 2.51 (4H, m under DMSO-d-5 signal) and 1.67 (4H, m); MS: (ES+ve) m/z 419, 421 [M+H]⁺

The amide, 4-bromo-N-[3-methoxy-4(2-pyrrolidin-1-yl-ethoxy)-phenyl]-benzamide (0.1 mM 42 mg), and 4-methyl-benzene boronic acid [Aldrich] (0.1 mM 14 mg) were refluxed for 16 hours in a mixture of benzene (8 ml), ethanol (2 ml) and 2M aqueous sodium carbonate (2 ml) in the presence of tetrakis-(triphenylphosphine)-palladium[0] (5 mg) under an argon atmosphere. The mixture was cooled, the upper layer decanted, and this solution purified by chromatography on silica gel using dichloromethane: methanol (10:1) followed by acetonitrile: satd. aqueous ammonia (25:1) to afford the title compound as a white solid.

¹H NMR (CDCl₃): δ 7.92 (2H, dd), 7.68 (2H, dd), 7.50 (21H, dd), 7.26 (3H, dddd), 6.96 (1H, dd), 6.88 (1H, d), 4.13 (1H, t), 3.87 (3H, s), 2.92 (2H, t), 2.60 (4H, m), 2.41 (3H, s) and 1.80 (4H, m); MS: (AP−ve) m/z 429 [M−H]⁻; (AP+ve) m/z 431 [M+H]⁺.

### EXAMPLE A100

Utilising the procedure of Example A93 with 4-(2,6-dimethoxypyrimidinyl)-boronic acid [Frontier] in place of 4-methyl-benzene boronic acid.

### EXAMPLE A103

Correspondingly Example A93 with furan-3-boronic acid [Frontier].

### EXAMPLE A104

Correspondingly Example A93 with mesityl-boronic acid [Frontier].

### EXAMPLE A105

Utilising the procedure of Example A51 except employing chloroform in place of dichloromethane as a solvent and eluent and utilising 3-quinuclidinol [Aldrich] in place of 1-(2-hydroxyethylpyrrolidine)

### EXAMPLE A107

Utilising the procedure of Example B37 except using piperidine in place of aniline.

### EXAMPLE B1

Utilising the procedure of Example A7 with 3-phenoxybenzoic acid [Aldrich] in place of 2′-methyl-biphenyl-4-carboxylic acid.

### EXAMPLE B2

Correspondingly Example B1 using 4-benzylbenzoic acid [Apin].

### EXAMPLE B34

### Correspondingly Example B1 using 3-benzylbenzoic acid [Patent WO-9828268].

### EXAMPLE B35

Correspondingly Example B1 using 4-phenoxybenzoic acid [Aldrich].

### EXAMPLE B37

N-[-[3-Methoxy-4-(2-pyrrolidin-1-yl-ethoxy)-phenyl]4-phenylamino-benzamide

Dry cesium carbonate (0.15 mM, 49 mg), (S)-BINAP [Aldrich] (0.015 mM, 9 mg) and palladium acetate (0.0075 mM, 2 mg) were sonicated in anhydrous ethyleneglycol dimethyl ether (15 ml) for 40 minutes under an argon atmosphere. This suspension was treated with 4-bromo-N-[3-methoxy-4-(2-pyrrolidin-1-yl-ethoxy)-phenyl]-benzamide [Example A93] (0.1 mM, 42 mg) and aniline (0.11 mM, 10 mg) then refluxed for 40 hours. The suspension was filtered through a hydrophobic membrane, concentrated, then purified on C18 R.P. silica using acetonitrile:water to afford the title compound as a white solid.

¹H NMR (MeOH-d₄): δ 7.96 (2H, dd) 7.92 (1H, d), 3.1 (2H, dd), 7.20 (1H, dd), 7.04 (1H, d), 4.28 (2H, t), 3.92 (3H, s), 3.78 (2H, m), 3.60 (2H, t), 3.58-3.13 (6H, m) and 2.26-1.47 (10H, m); MS: (ES+ve) m/z 424 [M+H]+

### EXAMPLE C1

Utilising the procedure of Example A7 with 2-methylbiphenyl-4-carboxylic acid [Patent WO-9606079] in place of 2′-methyl-biphenyl-4-carboxylic acid.

### EXAMPLE C2

Correspondingly Example C1 using 3-methoxybiphenyl-4-carboxylic acid [Patent WO-9534540].

### EXAMPLE C3

Correspondingly Example C1 using 3-methylbiphenyl-4-carboxylic acid [Patent WO-9534540].

### EXAMPLE C4

Correspondingly Example C1 using 4-phenylthiophene-2-carboxylic acid [Specs].

### EXAMPLE C5

Correspondingly Example C1 using 4-(3,5-dichlorophenoxy)-furan-2-carboxylic acid [Maybridge].

### EXAMPLE C6

Correspondingly Example C1 using 5-methyl-1-phenylpyrazole-4-carboxylic acid Maybridge].

### EXAMPLE C7

Correspondingly Example C1 using 6-phenyl-nicotinic acid [WO-0006085].

### EXAMPLE C8

Correspondingly Example C1 using 3-chloro-biphenyl-4-carboxylic acid [Patent JP-09221476].

### EXAMPLE C9

Correspondingly Example C1 using 5-(4-chlorophenyl)-2-trifluoromethyl-furan-3-carboxylic acid [Maybridge].

### EXAMPLE C10

Correspondingly Example C1 using 2-(4-chlorophenyl)-3-(trifluoromethyl)-pyrazole-4-carboxylic acid [Maybridge].

### EXAMPLE C11

Correspondingly Example C1 using 5-(2-pyridyl)-thiophene-2-carboxylic acid [Maybridge].

### EXAMPLE C12

Correspondingly Example C1 using 5-(methyl-trifluoromethyl-2-H-pyrazol-3-yl)-thiophene-2-carboxylic acid [Maybridge].

### Example D1

Utilising the procedure of Example D5 with 3,4-dichloronitrobenzene [Aldrich] in place of 2,4-dichloronitrobenzene.

### EXAMPLE D5

Biphenyl-4-carboxylic acid [2-chloro-4-(2-diisopropylamino-ethoxy)-phenyl]-amide.

To a three-neck flask (fitted with condenser, dropping funnel and thermometer) containing iron powder (938 mg, 16.8 mmol) mixed with a solution of ammonium chloride (28 mmol) in water (28 ml), was added the amine [2-(3-chloro-4-nitro-phenoxy)-ethyl]-diisopropyl-amine [prepared by the method used to form 1-[2-(2-methoxy-4-nitro-phenoxy)-ethyl]-pyrrolidine in Example.A51 but with 2-4-dichloronitrobenzene [Aldrich] in place of 4-chloro-3-methoxynitrobenzene and 2-diisopropylaminoethanol in place of 1-(2-hydroxyethyl)-pyrrolidine], dropwise over 10 minutes. The reaction mixture was gently refluxed until t.l.c. analysis showed no starting material. The mixture was filtered while hot and the inorganic residues washed with methanol. The combined filtrates were partitioned between water (5 ml) and ethyl acetate(3×10 ml), the organic phase dried (MgSO₄), filtered, and evaporated. The aqueous phase was treated with satd. aq. sodium bicarbonate (10 ml), extracted with ethyl acetate (3×10 ml), dried (MgSO₄), and evaporated. Residues from both extractions were combined and purified by flash chromatography on silica gel using dichloromethane—methanol—aq. ammonia as eluent to afford 2-chloro-4-(2-diisopropylamino-ethoxy)-phenylamine as a brown oil.

¹H NMR (CDCl₃): δ 1.02 (12H, d), 2.77 (2H, t), 3.03 (2H, sept.), 3.72 (2H, bs), 3.80 (2H, t), 6.68 (2H, m) and 6.85 (1H, m); MS (AP+ve): m/z 271, 273 [M+H]+.

This material was used in place of 3-methoxy-4-(2-pyrrolidin-1-yl-ethoxy)-phenylamine in the procedure of Example A51 to afford the title compound as clear oil.

¹H NMR (CDCl₃): δ 1.26 (12H, d), 3.07 (2H, m), 3.35 (2H, m), 4.22 (2H, m), 6.89 (1H, dd), 7.01 (1H, m), 7.44 (3H, q), 7.62 (2H, d), 7.71 (2H, d), 7.97 (2H, d) and 8.34 (1H, d); MS (AP+ve): m/z 452, 454 [M+H]⁺.

### EXAMPLE D9

Utilising the procedure of Example A51 with 2,4-difluoronitrobenzene [Aldrich] in place of 4-chloro-3-methoxynitrobenzene.

### EXAMPLE D12

### WO-00/061,461

Utilising the procedure of Example A51 with 3,4-difluoronitrobenzene [Aldrich] in place of 4-chloro-3-methoxynitrobenzene.

### EXAMPLE D16

Utilising the procedure of Example A51 with 2-methyl-4-fluoronitrobenzene [Aldrich] in place of 4-chloro-3-methoxynitrobenzene

### EXAMPLE D20

Utilising the procedure of Example A51 with 3-methyl-4-fluoronitrobenzene [Aldrich] in place of 4-chloro-3-methoxynitrobenzene

### EXAMPLE D24

Utilising the procedure of Example A51 with 3-acetyl-4-fluoronitrobenzene [Aldrich] in place of 4-chloro-3-methoxynitrobenzene

### EXAMPLE D25

Biphenyl-4-carboxylic acid [4-(2-diisopropylamino-ethoxy)-2-formyl-5-methoxy-phenyl]-amide

Biphenyl-4-carboxylic acid [4-(2-diisopropylamino-ethoxy)-3-methoxy-phenyl]-amide [Patent WO-9901127] (223 mg, 0.5 mmol) was treated with glyoxylic acid trihydrate (1 ml), dichloromethane (5 ml) and methanesulphonic acid (0.5 ml). The mixture was stirred vigorously for 24 hours then treated with satd. aq. sodium bicarbonate (30 ml) and extracted with dichloromethane (3×20 ml). The combined organic phases were dried (MgSO₄), filtered and evaporated, then subjected to flash chromatography on silica gel [chloroform—methanol—aqueous acetic acid] to obtain the title compound as the acetate salt, a white solid.

¹H NMR (CDCl₃): δ 1.13 (12H, d), 2.04 (3H, s), 3.02 (2H, t), 3.20 (2H, hept.), 4.05 (3H, s), 4.10 (2H, t), 5.0 (1H, bs), 7.22 (1H, s), 7.40 (1H, t), 7.48 (2H, d), 7.65 (2H, d), 7.76 (2H, d), 8.14 (2H, d), 8.72 (1H, s) and 9.34 (1H, s); MS (AP+ve): m/z 475 [M+H⁺].

### EXAMPLE D26

Biphenyl-4-carboxylic Acid [4-(2-diethylamino-ethoxy)-3-(1-hydroxy-ethyl)-phenyl]-amide

To biphenyl-4-carboxylic acid [3-acetyl-4-(2-diethylamino-ethoxy)-phenyl]-amide [Example D24] (20 mg, 0.05 mmol) dissolved in a 1:1 mixture of tetrahydrofuran/ethanol (3 ml), was added sodium borohydride [Aldrich] (6 mg, 0.15 mmol). The reaction mixture was stirred at ambient temperature for 16 hours. The solvent was evaporated and the residue purified by flash chromatography on silica gel using dichloromethane—aq. ammonia—methanol as eluents, to afford the title compound as a white solid.

¹H NMR (CDCl₃): δ 1.09 (6H, t), 1.49 (3H, d), 2.75 (4H, q), 2.95 (2H, t), 4.15 (2H, t), 5.01 (1H, q), 6.84 (1H, d), 7.45-7.67 (9H, m) and 7.95 (2H, d)

MS (AP+ve): m/z 433 [M+H⁺].

### EXAMPLE D27

Biphenyl-4-carboxylic acid [4-(2-diethylamino-ethoxy)-3-ethyl-phenyl]-amide

To biphenyl-4-carboxylic acid [3-acetyl-4-(2-diethylamino-ethoxy)-phenyl]-amide [Example D24] (25 mg, 0.06 mmol) dissolved in dichloromethane (1.5 ml), was added triethylsilane (0.5 ml) and trifluoroacetic acid (0.25 ml). The resulting yellow solution was stirred at room temperature for 120 h. The solvents were evaporated and the residue purified by flash chromatography on silica gel using dichloromethane—aq. ammonia—methanol as eluents to afford the title compound as white solid.

¹H NMR (CDCl₃): δ 1.17 (6H, m), 2.64 (2H, q), 2.8 (4H, q), 3.06 (2H, t), 4.15 (2H, t), 6.82 (1H, d), 7.35-7.71 (9H, m) and 7.96 (2H, d)

MS (AP+ve): m/z 417 [M+H]⁺

### EXAMPLE D28

### WO9901127

Utilising the procedure of Example A51 with 4-fluoronitrobenzene [Aldrich] in place of 4-chloro-3-methoxynitrobenzene, and 2-diisopropylaminoethanol in place of 1-(2-hydroxyethyl)-pyrrolidine

### EXAMPLE D30

### WO9901127

Utilising the procedure of Example D28 with 2-dimethylaminoethanol [Aldrich] in place of 2-diisopropylaminoethanol.

### EXAMPLE D32

### WO9901127

Utilising the procedure of Example D28 with 2-diethylaminoethanol [Aldrich] in place of 2-diisopropylaminoethanol

### EXAMPLE D38

### WO9901127

Utilising the procedure of Example A22 with 4-fluoronitrobenzene [Aldrich] in place of 4-chloro-3-methoxynitrobenzene, and 4-ethylphenylboronic acid in place of 4-methoxyphenylboronic acid

### EXAMPLE D39

### WO99011271

Utilising the procedure of Example A84 with 4-fluoronitrobenzene [Aldrich] in place of 4-chloro-3-methoxynitrobenzene, and 4-ethylphenylboronic acid in place of 4-methoxyphenylboronic acid.

### EXAMPLE E1

Biphenyl-4-carboxylic Acid [4-(2-diisopropylamino-ethoxy)-3-methoxy-phenyl]-methyl-amide.

To 4-(2-diisopropylamino-ethoxy)-3-methoxy-phenylamine (1 mmol) [Example A7] were added triethylorthoformate (8 ml) and trifluoroacetic acid (0.15 ml). The resulting solution was heated to 90° C. for 4 hr. The solution was evaporated then redissolved in ethanol and cooled to approximately −10° C. Sodium borohydride (190 mg, 5 mmol) was introduced portionwise over 10 minutes then the mixture allowed to warm to room temperature. The solution was stirred at room temperature for 16 h, then acidified to pH 1 with 2M hydrochloric acid. The mixture was concentrated to approximately 10 ml, then partitioned between ethyl acetate and water. The aqueous phase was adjusted to pH 14 using 2M aq sodium hydroxide solution, and extracted with dichloromethane (×3), dried (MgSO₄), filtered and evaporated. The residue was purified by flash chromatography on silica gel using dichloromethane—aq. ammonia—methanol as eluent to afford [4-(2-diisopropylamino-ethoxy)-3-methoxy-phenyl]-methyl-amine as an oil.

¹H NMR (CDCl₃): δ 1.03 (12H, d), 2.80 (3H, s), 2.85 (2H, t), 3.02 (2H, q), 3.80 (3H, s), 3.86 (2H, t), 6.13 (1H, dd), 6.23 (1H, d) and 6.80 (1H, d); MS (AP+ve): m/z 281[M+H]⁺.

To 4-phenylbenzoic acid (0.2 mmol) suspended in dichloromethane was added oxalyl chloride (0.6 mmol) followed by dimethylformamide (1 drop). The reaction mixture was stirred for 1 h, evaporated, co-evaporated (×3) with dichloromethane then redissolved in dichloromethane(1 ml). A solution containing the amine [4-(2-diisopropylamino-ethoxy)-3-methoxy-phenyl]-methyl-amine (0.2 mmol) and triethylamine (140 mg, 1 mmol) dissolved in dichloromethane (1 ml) was added. This solution was stirred at ambient temperature for 14 hours, evaporated, dissolved in dichloromethane (1 ml) and treated with PS-isocyanate resin [Argonaut Technologies] (150 mg). After a further 18 h shaking at ambient temperature, the mixture was filtered, passed through an SAX column [Varian] (1 g), evaporated, and the residue purified by chromatography on silica gel using dichloromethane—aq. ammonia—methanol as eluent to afford the title compound as an oil.

¹H NMR (CDCl₃): δ 1.21 (12H, bd), 2.88-3.24 (4H, m), 3.32 (3H, s), 3.87 (3H, s), 4.11 (2H, m), 6.82-6.91 (3H, m) and 7.26-7.56 (9H, m); MS (AP+ve): m/z 476 [M+M]⁺.

### EXAMPLE E5

Utilising the procedure of Example E1 with triethyl orthoacetate [Aldrich] in place of triethyl orthoformate.

### EXAMPLE E12

Biphenyl-4-carboxylic Acid [2-chloro-4-(2-diisopropylamino-ethoxy)-5-methoxy-phenyl]-amide

Biphenyl-4-carboxylic acid [4-(2-diethylamino-ethoxy)-3-methoxy-phenyl]-methyl-amide [Example E9] (45 mg, 0.1 mmol), was dissolved in chloroform (1 ml) and treated with benzotriazole [Aldrich] (12 mg, 0.1 mmol) and N-chlorosuccinimide (13 mg, 0.11 mmol). The mixture was stirred at ambient temperature for 16 hours then evaporated and subjected to flash chromatography on silica gel (dichloromethane—methanol—aqueous ammonia) to afford the title compound as an oil.

¹H NMR (CDCl₃): δ 1.06 (6H, t), 2.63 (4H, q), 2.90 (2H, t), 3.39 (3H, s), 3.67 (3H, s), 4.03 (2H, t), 6.57 (1H, s), 6.84 (1H, s) and 7.31-7.53 (9H, m); MS (AP+ve): m/z 467, 469 [M+H]⁺.

### EXAMPLE E13

Utilising the procedures of Example A93 with [4-(2-diethylamino-ethoxy)-3-methoxy-phenyl]-methyl-amine [Example E9] in place of 4-(2-diethylamino-ethoxy)-3-methoxy-phenylamine and 2-fluoromethylphenylboronic acid [Aldrich] in place of 4-methoxyphenylboronic acid and of Example 51 with (N-diethyl)ethanolamine in place of 1-(2-hydroxyethyl)pyrrolidine.

### EXAMPLE E14

Utilising the procedure of Example E13 with 2-methylphenylboronic [Aldrich] in place of of 4-chlorophenylboronic acid.

### EXAMPLE E16

Correspondingly Example E14 with 2-chloromethylphenylboronic acid [Aldrich].

### EXAMPLE E17

Correspondingly Example E14 with 4-fluoromethylphenylboronic acid [Aldrich].

### EXAMPLE E21

Correspondingly Example E14 with 4-chloromethylphenylboronic acid [Aldrich].

### EXAMPLE E22

Correspondingly Example E14 with 4-ethylphenylboronic acid [Aldrich].

### EXAMPLE E23

Correspondingly Example E14 with 4-tertbutylphenylboronic acid [Aldrich].

### EXAMPLE E24

4-Biphenylcarboxylic acid [4-(2-diethylamino-ethoxy)-3-methoxy-phenyl]-methyl-amide [Example E9] (45 mg, 0.1 mmol), was dissolved in acetonitrile (1 ml) and treated with N-fluoro-N′-chloromethyl-triethylenediamine-bis(tetrafluoroborate) (43 mg, 0.12 mmol) and heated to 80° C. for 6 hours. The solvent was evaporated and the residue subjected to flash chromatography on silica gel (dichloromethane—methanol—aqueous ammonia) to afford the title compound as an oil.

MS (AP+ve): m/z 451 [M+H]⁺.

### EXAMPLE E25

Utilising the procedure of Example E1 with 4-(2-diisopropylamino-ethoxy)-3-methyl-phenylamine [Example D20] in place of 4-(2-diisopropylamino-ethoxy)-3-methoxy-phenylamine and triethyl orthoacetate in place of triethyl orthoformate.

### EXAMPLE F1

Utilising the procedure of Example A7 with 6-phenyl-nicotinic acid (Patent WO-0006085) in place of 2′-methyl-4-biphenylcarboxylic acid and N-dimethylethanolamine in place of 2-(diisopropylamino)ethanol.

### EXAMPLE G1

Biphenyl-4-carboxylic acid [4-((R)-diethylamino-hydroxy-propoxy)-3-methoxy-phenyl]-amide

4-Nitro-2-methoxyphenol [Aldrich] (845 mg, 5 mmol) was dissolved in DMF (25 ml) and treated with sodium hydride (60% oil dispersion, 200 mg). When the effervescence ceased, the mixture was treated with (R)-p-nitrophenylsulphonyl glycidol [Aldrich] and warmed to 50° C. with stirring. After 16 hours, the mixture was cooled, evaporated, partitioned between water (20 ml) and dichloromethane (3×25 ml), dried (MgSO₄), filtered and evaporated. The residue was purified by flash chromatography on silica gel (hexane—ether) to give (R)-2-(2-methoxy-4-nitro-phenoxymethyl)-oxirane as a pale brown solid in 80% yield.

¹H NMR (CDCl₃): δ 2.79 (1H, dd), 2.95 (1H, dd), 3.41 (1H, dddd), 3.96 (3H, s), 4.06 (1H, dd), 4.43 (1H, dd), 6.98 (1H, d), 7.75 (1H, d) and 7.87 (1H, dd).

(R)-2-(2-Methoxy-4-nitro-phenoxymethyl)-oxirane (0.5 mmol, 113 mg), in dichloromethane (3 ml) was treated with the amine (diethylamine) [Aldrich] (1.5 mmol, 110 mg) and titanium tetraisopropoxide [Aldrich] (50 ul). The solution was stirred at ambient temperature for 24 h, treated with water (1 ml) and shaken vigorously for 10 minutes. The resulting suspension was passed through a hydromatrix cartridge [Varian ChernElut] (5 ml) eluting with dichloromethane (10 ml) to give (R)-diethylamino-(2-methoxy-4-nitro-phenoxy)-propan-2-ol as a yellow oil

¹H NMR (CDCl₃): δ 1.07 (6H, t), 2.55-2.72 (7H, m), 3.94 (3H, s), 4.09-4.13 (3H, m), 6.97 (1H, d), 7.74 (1H, d) and 7.89 (1H, dd); MS (AP+ve): m/z 299 [M+H⁺].

This material was dissolved in ethanol (5 ml) and treated with hydrogen chloride (2M in diethyl ether) 0.1 ml then 10% palladium on charcoal (20 mg) and hydrogenated at atmospheric pressure for 24 hours. The solution was purged with argon then filtered through celite and evaporated to give (R)-(4-amino-2-methoxy-phenoxy)-diethylamino-propan-2-ol hydrochloride as a white crystalline solid.

¹H NMR (CD₃OD): δ 1.19 (6H, t), 3.36-3.45 (6H, m), 3.88 (s, 3H), 4.02-4.11 (2H, m), 4.03 (1H, m), 6.95-7.03 (2H, m) and 7.13 (1H, d).

A solution of this material in dichloromethane (2 ml) was treated with triethylamine (2 mmol, 280 ul) and triethylsilyl trifluoromethanesulphonate (1 mmol, 264 mg). After 30 minutes, 4-biphenylcarboxylic acid chloride [Example 1] (1 mmol, 217 mg) was introduced and the mixture stirred for 12 hours. The solvent was evaporated and the residue dissolved in methanol (100 ml) and treated with potassium carbonate (2 g). After stirring for six hours, the suspension was evaporated, formed into a slurry with dichloromethane (20 ml), filtered, the filtrate evaporated, and the residue purified by flash chromatography (dichloromethane—methanol—aq. ammonia) to give the title compound as a white solid.

¹H NMR (CDCl₃): δ 1.11 (6H, t), 2.61-2.78 (6H, m,), 3.88 (3H, s), 3.54.5 (1H, vbs), 3.99-4.13 (3H, m), 6.92 (1H, d), 6.99 (1H, dd), 7.41-7.49 (3H, m), 7.56 (1H, d), 7.63 (2H, d), 7.69 (2H, d) and 7.97 (3H, d); MS (AP+ve): m/z 449 [M+H⁺].

### EXAMPLE G5

Utilising the procedure for the preparation of (R)-diethylamino-(2-methoxy-4-nitro-phenoxy)-propan-2-ol [Example G1] but replacing dichloromethane with 1,2-dichloroethane and diethylamine with diisopropylamine. In addition, the mixture of amine and epoxide was heated at 80° C. for 12 h rather than being kept at ambient temperature for 24 hours.

### EXAMPLE G8

Utilising the procedure of Example G1 but using (S)-p-nitrophenylsulphonyl-glycidol in place of (R)-p-nitrophenylsulphonyl-glycidol, and pyrrolidine in place of diethylamine.

### EXAMPLE G22

Utilising the procedure of Example A51 but using 4-dimethylamino-1-butanol [ICN-RF] in place of 1-(2-hydroxyethyl)-pyrrolidine.

### EXAMPLE H1

4-Cyclohexyl-N-[3-methoxy-4-(4-methyl-piperazin-1-yl)-phenyl]-benzamide

A solution of 1-(2-methoxy-4-nitro-phenyl)-piperazine (Patent WO-9906382) (10 mmol, 2.37 g) in dichloromethane (50 ml) was treated with ditertbutyl dicarbonate (10 mmol, 2.18 g) with stirring. Vigorous evolution of gas occurred which ceased after 1 hour. The solution was then evaporated to a yellow solid 4-(2-methoxy-4-nitro-phenyl)-piperazine-1-carboxylic acid tertbutyl ester.

¹H NMR (CDCl₃): δ 1.50 (9H, s), 3.16 (4H, t), 3.61 (4H, t), 3.96 (3H, s), 6.88 (1H, d), 7.72 (1H, d) and 7.86 (1H, dd).

This material was dissolved in ethanol (50 ml) and treated with 10% Pd on carbon (100 mg). The suspension was hydrogenated at 1 atmosphere for 2 hours, then filtered through celite and evaporated to give 4-(4-amino-2-methoxy-phenyl)-piperazine-1-carboxylic acid tertbutyl ester as a brown oil.

¹H NMR (CDCl₃): δ 1.48 (9H, s), 2.86-2.91 (4H, t), 3.52-3.60 (4H, t), 3.81 (3H, s), 6.22-6.27 (2H, m) and 6.73 (1H, d).

This aniline (0.2 mmol, 61 mg) was dissolved in dichloromethane (1 ml) and treated successively with DIEA resin [Argonaut Technologies] (0.5 g) and 4-cyclohexylbenzoyl chloride [Example A36]. The mixture was shaken gently for 12 hours then filtered, evaporated and the residue purified by flash chromatography on silica gel (dichloromethane—methanol—aq. ammonia) to afford 4-(4-{[1-(4-cyclohexyl-phenyl)-methanoyl]-amino}-2-methoxy-phenyl)-piperazine-1-carboxylic acid tertbutyl ester as a white crystalline solid

¹H NMR (CDCl₃): δ 1.25-1.47 (5H, m), 1.54 (9H, s), 1.75-1.88 (5H, m), 2.56 (1H, m), 2.98 (4H, t), 3.61 (4H, t), 3.91 (3H, s), 6.87 (1H, d), 6.93 (1H, dd), 7.32 (2H, d), 7.54 (1H, s), 7.77, (1H, s) and 7.78 (2H, d); MS (AP+ve): m/z 493 [M+H⁺].

This material was dissolved in dichloromethane (5 ml) and treated with anisole (1 ml) and trifluoroacetic acid (5 ml). After 2 hours the solution was evaporated, then co-evaporated twice from toluene. The residue was dissolved in dichloromethane (10 ml), washed with satd. sodium bicarbonate (2 ml), the organic phase dried (MgSO₄), filtered and evaporated to a brown oil, 4-cyclohexyl-N-(3-methoxy-4-piperazin-1-yl-phenyl)-benzamide.

¹H NMR (CDCl₃): δ 1.22-1.87 (10, m), 2.57 (1, m), 3.04-3.12 (8H, m), 3.91 (3H, s), 6.95 (2H, bs), 7.32 (2H, d), 7.54 (1H, m), 7.77 (1H, s) and 7.78 (2H, d); MS (AP+ve): m/z 394 [M+H⁺].

This amine (0.1 mmol, 39 mg) was dissolved in ethanol (3 ml) and treated with metaformaldehyde (100 mg), Amberlyst cyanoborohydride resin [Novabiochem] (100 mg), and acetic acid (50 ul). The mixture was stirred at ambient temperature for three hours then filtered, evaporated and the residue purified by flash chromatography on silica gel (dichloromethane—methanol—aq. ammonia) to afford the title compound as a pale brown oil. This was evaporated from dilute acetic acid to give the monoacetate salt hydrate.

¹H NMR (CDCl₃): δ 1.22-1.45 (5H, m), 1.76-1.87 (5H, m), 2.02 (6H, 2×s), 2.56 (1H, m), 3.22-3.23 (4H, t), 3.29-3.30 (4H, t), 3.88 (3H, s), 6.86 (1H, d), 6.94 (1H, dd), 7.30 (1H, d), 7.59 (1H, d), 7.79 (2H, d), 7.98 (1H, s) and 8.54 (4H, bs);

MS (AP+ve): m/z 408 [M+H⁺].

The following tables give Examples which illustrate but do not limit the invention in any way.

TABLE A
Encompassing compounds of general formula (II), a subset of formula (I) where A = H
and OMe, R3 = H, X = O, Y = CH₂CH₂, Z = a bond; R4 = Ph and R5 is either meta or
para substituted on R4.

| | |
|---|---|
| | (II) |
| 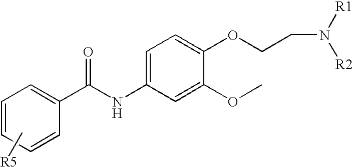 | |

| Example No. | R5 | 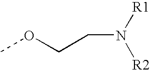 | meta/ para | [M + H]⁺ | Procedure |
|---|---|---|---|---|---|
| A1 | Ph | 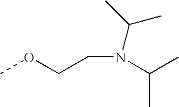 | p | 447 | A7 |
| A2 | 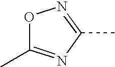 | 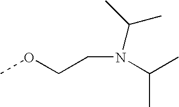 | p | 453 | A7 |
| A3 | 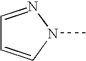 | 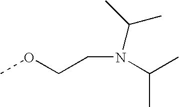 | p | 437 | A7 |
| A4 | Ph | 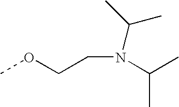 | m | 447 | A7 |
| A5 | 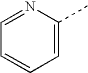 | 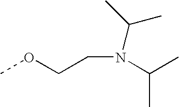 | p | 448 | A7 |
| A6 | 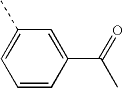 | 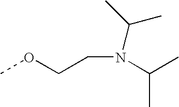 | p | 489 | A7 |
| A7 | 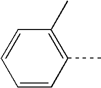 | 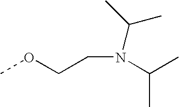 | p | 461 | A7 |
| A8 | 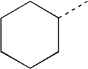 | 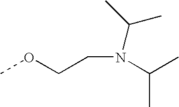 | p | 453 | A7 |
| A9 | 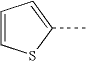 | 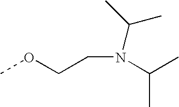 | p | 453 | A7 |
| A10 | 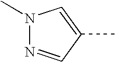 | 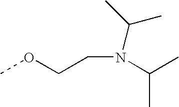 | p | 451 | A7 |
| A11 | 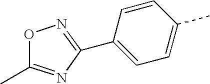 | 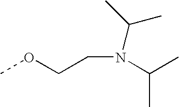 | p | 529 | A7 |
| A12 | 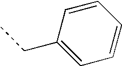 | 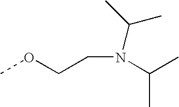 | p | 461 | A7 |
| A13 | 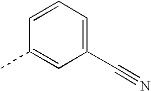 | 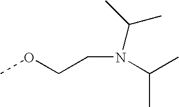 | m | 472 | A7 |
| A14 | 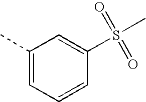 | 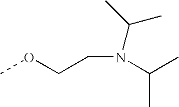 | p | 525 | A7 |
| A15 | 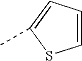 | 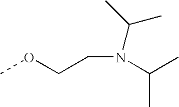 | m | 453 | A7 |
| A16 | 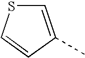 | 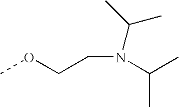 | m | 453 | A7 |
| A17 | 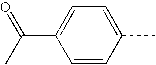 | 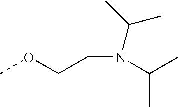 | p | 489 | A7 |
| A18 | 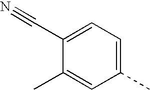 | 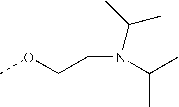 | p | 486 | A7 |
| A19 | 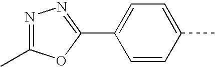 | 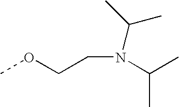 | p | 529 | A7 |
| A20 | 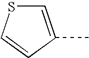 | 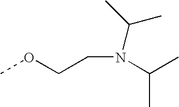 | p | 453 | A7 |
| A21 | 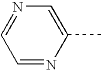 | 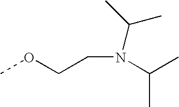 | p | 449 | A7 |
| A22 | 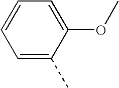 | 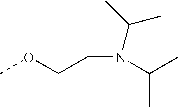 | p | 477 | A22 |
| A23 | 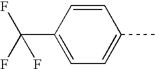 | 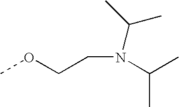 | p | 515 | A22 |
| A24 | 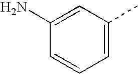 | 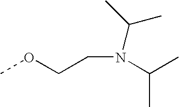 | p | 462 | A22 |
| A25 | 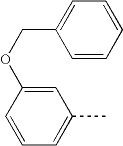 | 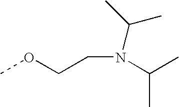 | p | 553 | A22 |
| A26 | 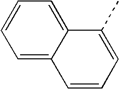 | 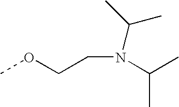 | p | 497 | A22 |
| A27 | 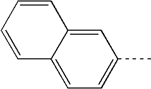 | 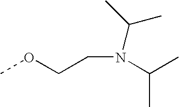 | p | 497 | A22 |
| A28 | 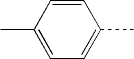 | 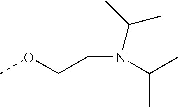 | p | 461 | A22 |
| A29 | 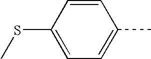 | 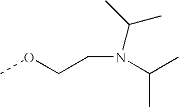 | p | 493 | A22 |
| A30 | 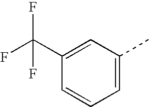 | 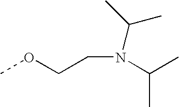 | p | 515 | A22 |
| A31 | 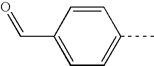 | 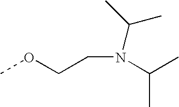 | p | 475 | A22 |
| A32 | 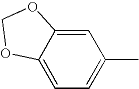 | 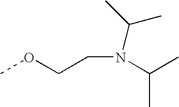 | p | 491 | A22 |
| A33 | 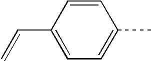 | 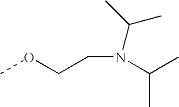 | p | 473 | A22 |
| A34 | 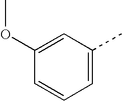 | 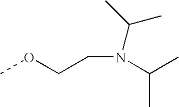 | p | 477 | A22 |
| A35 | Ph | 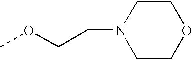 | p | 433 | A51 |
| A36 | 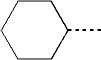 | 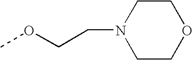 | p | 439 | A51 |
| A37 | Ph | 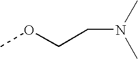 | p | 397 | A51 |
| A38 | 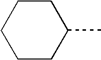 | 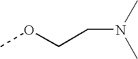 | p | 391 | A51 |
| A39 | 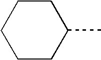 | 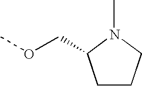 | p | 423 | A51 |
| A40 | Ph | 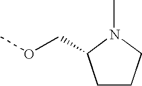 | p | 417 | A51 |
| A41 | Ph | 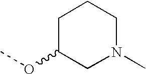 | p | 417 | A51 |
| A42 | 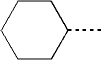 | 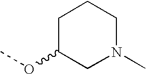 | p | 423 | A51 |
| A43 | Ph | 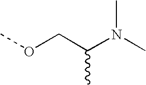 | p | 405 | A51 |
| A44 | 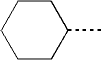 | 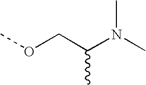 | p | 411 | A51 |
| A45 | Ph | 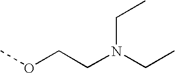 | p | 419 | A51 |
| A46 | 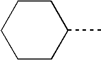 | 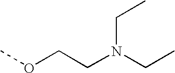 | p | 425 | A51 |
| A47 | Ph | 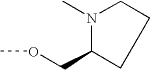 | p | 417 | A51 |
| A48 | 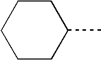 | 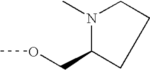 | p | 423 | A51 |
| A49 | Ph | 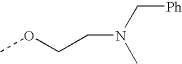 | p | 467 | A51 |
| A50 | 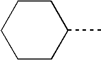 | 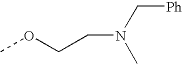 | p | 473 | A51 |
| A51 | Ph | 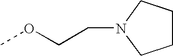 | p | 417 | A51 |
| A52 | 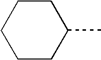 | 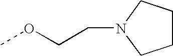 | p | 423 | A51 |
| A53 | 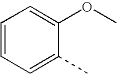 | 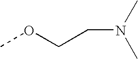 | p | 421 | A22 |
| A54 | Ph | 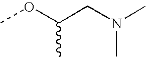 | p | 405 | A51 |
| A55 | 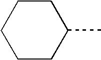 | 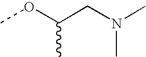 | p | 411 | A51 |
| A56 | Ph | 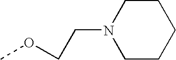 | p | 431 | A51 |
| A57 | 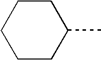 | 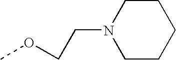 | p | 437 | A51 |
| A58 | Ph | 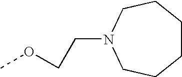 | p | 445 | A51 |
| A59 | 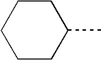 | 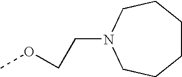 | p | 451 | A51 |
| A60 | 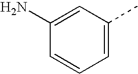 | 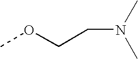 | p | 406 | A60 |
| A61 | 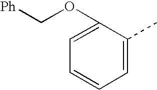 | 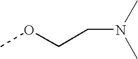 | p | 497 | A63 |
| A62 | 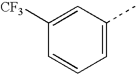 | 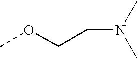 | p | 459 | A63 |
| A63 | 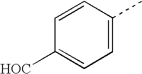 | 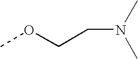 | p | 419 | A63 |
| A64 | 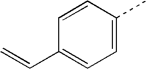 | 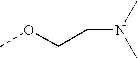 | p | 417 | A63 |
| A65 | 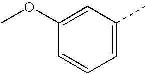 | 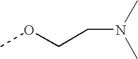 | p | 421 | A63 |
| A66 | 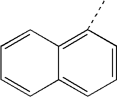 | 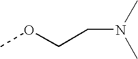 | p | 441 | A63 |
| A67 | 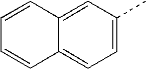 | 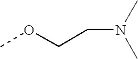 | p | 441 | A63 |
| A68 | 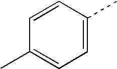 | 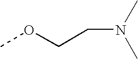 | p | 404 | A63 |
| A69 | 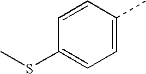 | 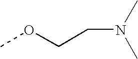 | p | 437 | A63 |
| A70 | 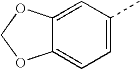 | 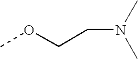 | p | 434 | A63 |
| A71 | 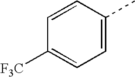 | 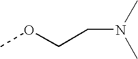 | p | 459 | A63 |
| A72 | Ph | 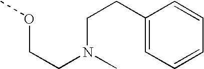 | p | 481 | A51 |
| A73 | 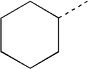 | 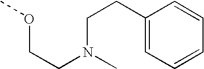 | p | 487 | A51 |
| A74 | Ph | 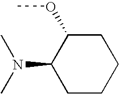 | p | 445 | A51 |
| A75 | 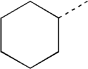 | 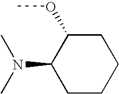 | p | 451 | A51 |
| A76 | Ph | 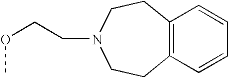 | p | 493 | A51 |
| A77 | 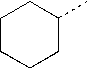 | 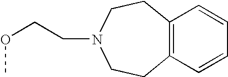 | p | 499 | A51 |
| A78 | Ph | 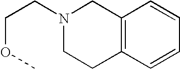 | p | 479 | A51 |
| A79 | 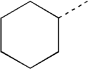 | 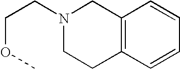 | p | 485 | A51 |
| A80 | Ph | 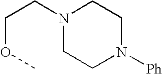 | p | 508 | A51 |
| A81 | 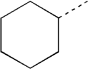 | 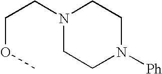 | p | 514 | A51 |
| A82 | Ph | 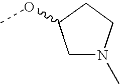 | p | 403 | A51 |
| A83 | 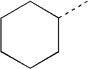 | 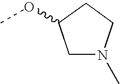 | p | 409 | A51 |
| A84 | 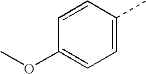 | 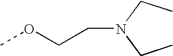 | p | 449 | A84 |
| A85 | 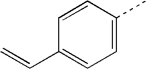 | 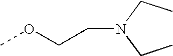 | p | 445 | A88 |
| A86 | 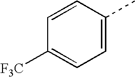 | 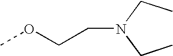 | p | 487 | A88 |
| A87 | 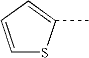 | 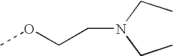 | p | 425 | A88 |
| A88 | 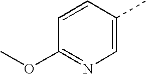 | 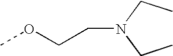 | p | 450 | A88 |
| A89 | 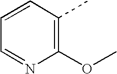 | 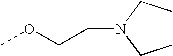 | p | 450 | A88 |
| A90 | 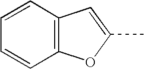 | 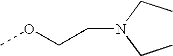 | p | 459 | A88 |
| A91 | 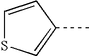 | 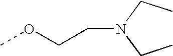 | p | 425 | A88 |
| A92 | 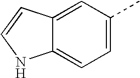 | 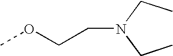 | p | 458 | A88 |
| A93 | 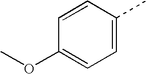 | 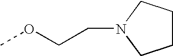 | p | 447 | A93 |
| A94 | 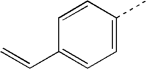 | 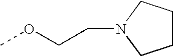 | p | 443 | A93 |
| A95 | 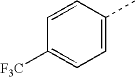 | 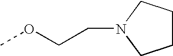 | p | 485 | A93 |
| A96 | 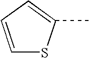 | 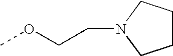 | p | 423 | A93 |
| A97 | 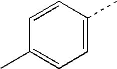 | 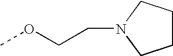 | p | 431 | A93 |
| A98 | 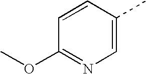 | 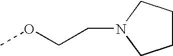 | p | 448 | A93 |
| A99 | 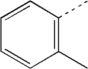 | 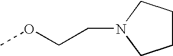 | p | 431 | A93 |
| A100 | 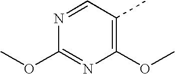 | 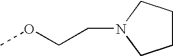 | p | 479 | A93 |
| A101 | 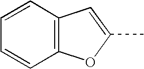 | 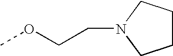 | p | 457 | A93 |
| A102 | 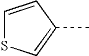 | 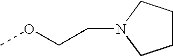 | p | 423 | A93 |
| A103 | 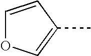 | 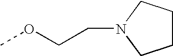 | p | 407 | A93 |
| A104 | 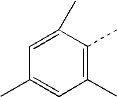 | 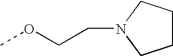 | p | 459 | A93 |
| A105 | Ph | 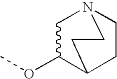 | p | 429 | A105 |
| A106 | 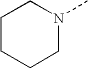 | 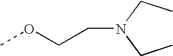 | p | 426 | A107 |
| A107 | 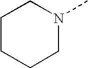 | 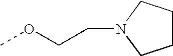 | p | 424 | A107 |
| A108 | 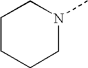 | 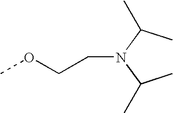 | p | 454 | A107 |

TABLE B
Encompassing compounds of general formula (III), a subset of formula (1)
where A = H and OMe, R1 = R2 = Me₂, R3 = H,
X = O, Y = CH₂—CH₂, Z = O, CH₂ or NH; R4 = Ph,
R5 is Ph and Z is either meta or para substituted on R4.

| | |
|---|---|
| | (III) |
| 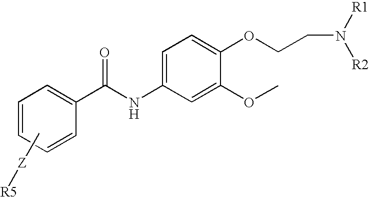 | |

| Example No. | Z | meta/ para | 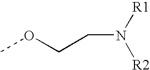 | [M + H]⁺ | Procedure |
|---|---|---|---|---|---|
| B1 | O | m | 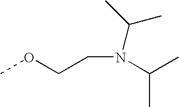 | 463 | B1 |
| B2 | CH₂ | p | 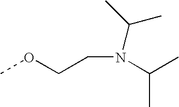 | 461 | B1 |
| B3 | O | m | 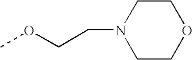 | 229 | A51 |
| B4 | CH₂ | p | 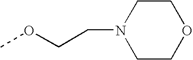 | 4447 | A51 |
| B5 | O | m | 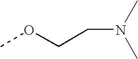 | 407 | A51 |
| B6 | CH₂ | p | 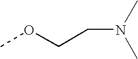 | 405 | A51 |
| B7 | O | m | 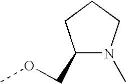 | 433 | A51 |
| B8 | CH₂ | p | 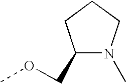 | 431 | A51 |
| B9 | O | m | 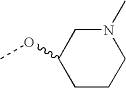 | 433 | A51 |
| B10 | CH₂ | p | 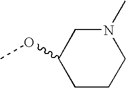 | 431 | A51 |
| B11 | O | m | 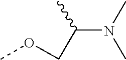 | 421 | A51 |
| B12 | CH₂ | p | 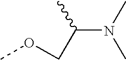 | 419 | A51 |
| B13 | O | m | 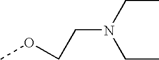 | 435 | A51 |
| B14 | CH₂ | p | 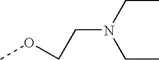 | 433 | A51 |
| B15 | O | m | 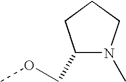 | 433 | A51 |
| B16 | CH₂ | p | 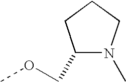 | 431 | A51 |
| B17 | O | m | 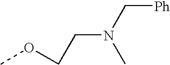 | 483 | A51 |
| B18 | CH₂ | p | 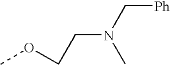 | 481 | A51 |
| B19 | O | m | 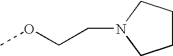 | 433 | A51 |
| B20 | CH₂ | p | 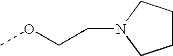 | 431 | A51 |
| B21 | CH₂ | p | 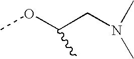 | 419 | A51 |
| B22 | O | m | 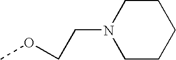 | 447 | A51 |
| B23 | CH₂ | p | 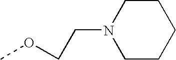 | 445 | A51 |
| B24 | O | m | 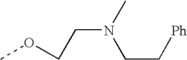 | 497 | A51 |
| B25 | CH₂ | p | 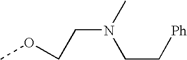 | 495 | A51 |
| B26 | O | m | 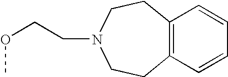 | 509 | A51 |
| B27 | CH₂ | p | 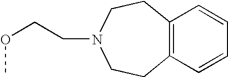 | 507 | A51 |
| B28 | O | m | 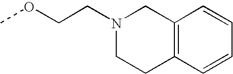 | 495 | A51 |
| B29 | CH₂ | p | 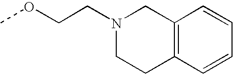 | 493 | A51 |
| B30 | O | m | 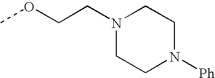 | 524 | A51 |
| B31 | CH₂ | p | 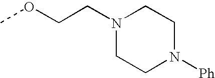 | 522 | A51 |
| B32 | O | m | 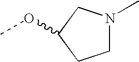 | 419 | A51 |
| B33 | CH₂ | p | 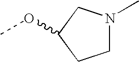 | 417 | A51 |
| B34 | CH₂ | m | 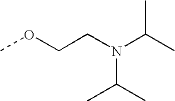 | 461 | B1 |
| B35 | O | p | 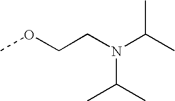 | 463 | B1 |
| B36 | NH | p | 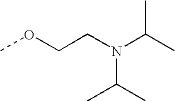 | 462 | B37 |
| B37 | NH | p | 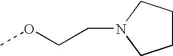 | 432 | B37 |

TABLE C
Encompassing compounds of general formula (IV) a subset of formula (1)
where A = H and OMe, R1 = R2 = Me₂, R3 = H, X = O,
Y = CH₂—CH₂; R4, R5 = substituted phenyl or heterocycle,

| | |
|---|---|
| | (IV) |
| 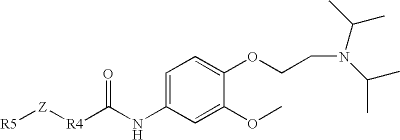 | |

| Example No. | Z | 3/4 substitution w.r.t C═O | 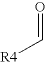 | 10 R5 | [M + H]⁺ | Method |
|---|---|---|---|---|---|---|
| C1 | bond | 4 | 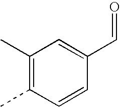 | Ph | 461 C1 | |
| C2 | bond | 4 | 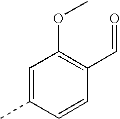 | Ph | 477 | C1 |
| C3 | bond | 4 | 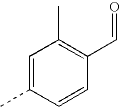 | Ph | 461 | C1 |
| C4 | bond | 3 | 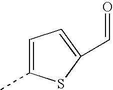 | Ph | 453 | C1 |
| C5 | O | 3 | 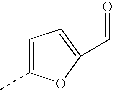 | 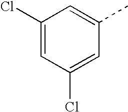 | 521, 523, 525 | C1 |
| C6 | bond | 3 | 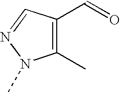 | Ph | 451 | C1 |
| C7 | bond | 4 | 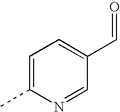 | Ph | 448 | C1 |
| C8 | bond | 4 | 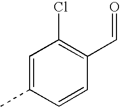 | Ph | 481, 483 | C1 |
| C9 | bond | 3 | 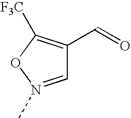 | 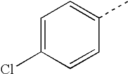 | 539, 541 | C1 |
| C10 | bond | 3 | 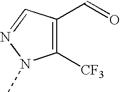 | 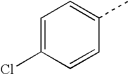 | 539 | C1 |
| C11 | bond | 3 | 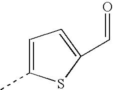 | 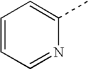 | 453 | C1 |
| C12 | bond | 3 | 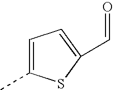 | 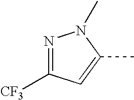 | 525 | C1 |

TABLE D
Encompassing compounds of general formula (V) a subset of formula (I)
where R3 = H, X = O, Y = CH₂—CH₂, Z = O, CH₂, NH or a bond;
R4 = Ph, R5 is Ph or cyclohexyl (Cy) and Z is either meta or para
substituted on R4.

| | |
|---|---|
| | (V) |
| 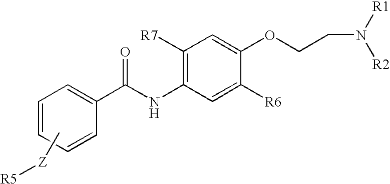 | |

| Example No. | Z | R6 | R7 | R5 | meta/ para | 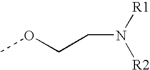 | [M + H]⁺ | Method |
|---|---|---|---|---|---|---|---|---|
| D1 | bond | Cl | H | Ph | p | 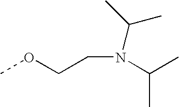 | 452, 454 | D1 |
| D2 | O | Cl | H | Ph | m | 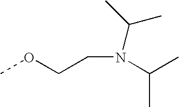 | 468, 470 | D1 |
| D3 | CH₂ | Cl | H | Ph | p | 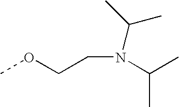 | 466, 468 | D1 |
| D4 | bond | Cl | H | Cy | p | 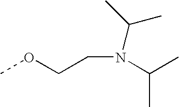 | 458, 460 | D1 |
| D5 | bond | H | Cl | Ph | p | 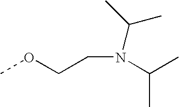 | 452, 454 | D5 |
| D6 | O | H | Cl | Ph | m | 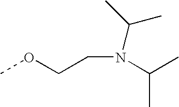 | 468, 470 | D5 |
| D7 | CH₂ | H | Cl | Ph | p | 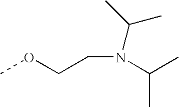 | 466, 468 | D5 |
| D8 | bond | H | Cl | Cy | p | 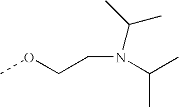 | 458, 460 | D5 |
| D9 | bond | F | H | Ph | p | 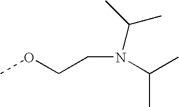 | 435 | D9 |
| D10 | CH₂ | F | H | Ph | p | 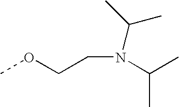 | 449 | D9 |
| D11 | bond | F | H | Ph | p | 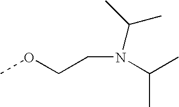 | 441 | D9 |
| D12 | bond | H | F | Ph | p | 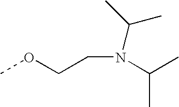 | 435 | D12 |
| D13 | O | H | F | Ph | m | 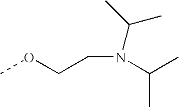 | 451 | D12 |
| D14 | CH₂ | H | F | Ph | p | 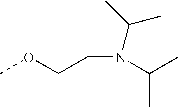 | 449 | D12 |
| D15 | bond | H | F | Cy | p | 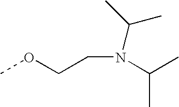 | 441 | D12 |
| D16 | bond | Me | H | Ph | p | 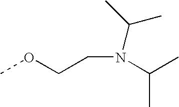 | 431 | D16 |
| D17 | O | Me | H | Ph | m | 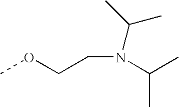 | 447 | D16 |
| D18 | CH₂ | Me | H | Ph | p | 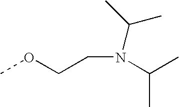 | 445 | D16 |
| D19 | bond | Me | H | Cy | p | 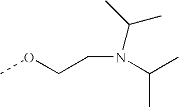 | 437 | D16 |
| D20 | bond | H | Me | Ph | p | 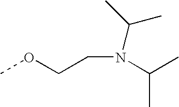 | 431 | D20 |
| D21 | O | H | Me | Ph | m | 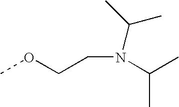 | 447 | D20 |
| D22 | CH₂ | H | Me | Ph | p | 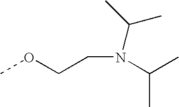 | 445 | D20 |
| D23 | bond | H | Me | Cy | p | 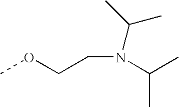 | 437 | D20 |
| D24 | bond | COCH₃ | H | Ph | p | 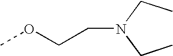 | 431 | D24 |
| D25 | bond | OMe | CHO | Ph | p | 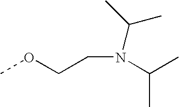 | 475 | D25 |
| D26 | bond | CH(OH)CH₃ | H | Ph | p | 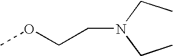 | 433 | D26 |
| D27 | bond | Et | H | Ph | p | 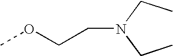 | 417 | D27 |
| D28 | bond | H | H | Ph | p | 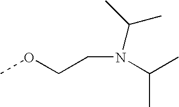 | 417 | D28 |
| D29 | O | H | H | Ph | m | 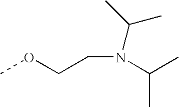 | 433 | D28 |
| D30 | bond | H | H | Ph | p | 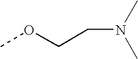 | 361 | D30 |
| D31 | O | H | H | Ph | p | 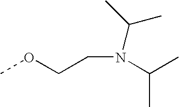 | 433 | D28 |
| D32 | O | H | H | Ph | p | 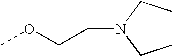 | 405 | D32 |
| D33 | O | H | H | Ph | m | 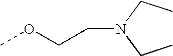 | 405 | D32 |
| D34 | bond | H | H | Cy | p | 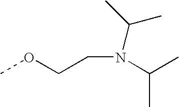 | 423 | D28 |
| D35 | bond | H | H | Cy | p | 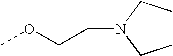 | 395 | D32 |
| D36 | CH₂ | H | H | Ph | p | 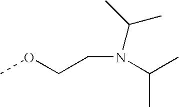 | 431 | D28 |
| D37 | CH₂ | H | H | Ph | p | 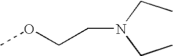 | 403 | D32 |
| D38 | bond | H | H | p-EtPh | p | 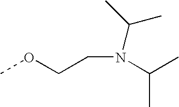 | 445 | D38 |
| D39 | bond | H | H | p-EtPh | p | 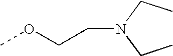 | 417 | D39 |

TABLE E
Encompassing compounds of general formula (VI) a subset of formula (1)
where A = H, Cl, F and OMe, X = O, Y = CH₂—CH₂; R4 = phenyl,
R5 = phenyl or cyclohexyl (Cy), Z = O, CH₂ or a bond

| | |
|---|---|
| | (VI) |
| 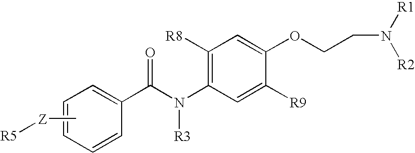 | |

| Example No. | Z | o/p | R3 | R8 | R9 | R5 | 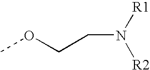 | [M + H]⁺ | Method |
|---|---|---|---|---|---|---|---|---|---|
| E1 | bond | p | Me | H | MeO | Ph | 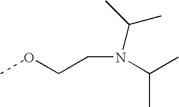 | 461 | E1 |
| E2 | O | m | Me | H | MeO | Ph | 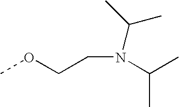 | 477 | E1 |
| E4 | CH₂ | p | Me | H | MeO | Ph | 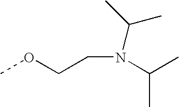 | 475 | E1 |
| E5 | bond | p | Me | H | MeO | Cy | 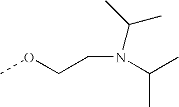 | 467 | E1 |
| E6 | bond | p | Et | H | MeO | Ph | 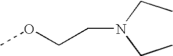 | 447 | E1 |
| E7 | bond | p | Et | H | MeO | Ph | 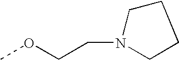 | 445 | E1 |
| E8 | bond | p | Me | H | MeO | Ph | 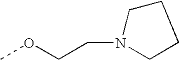 | 431 | E1 |
| E9 | bond | p | Me | H | MeO | Ph | 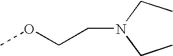 | 433 | E1 |
| E10 | bond | p | Et | H | MeO | Cy | 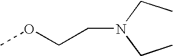 | 453 | E1 |
| E11 | bond | p | Et | H | MeO | Cy | 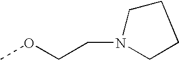 | 451 | E1 |
| E12 | bond | p | Me | Cl | MeO | Ph | 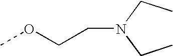 | 468, 470 | E12 |
| E13 | bond | P | Me | H | MeO | 2-F—Ph | 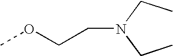 | 451 | E13 |
| E14 | bond | p | Me | H | MeO | 2-Me—Ph | 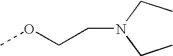 | 447 | E14 |
| E15 | bond | p | Me | H | MeO | 2-MeO—Ph | 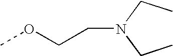 | 463 | E14 |
| E16 | bond | p | Me | H | MeO | 2-Cl—Ph | 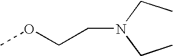 | 468, 470 | E14 |
| E17 | bond | p | Me | H | MeO | 4-F—Ph | 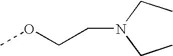 | 451 | E14 |
| E18 | bond | p | Me | H | MeO | 4-F₃C—Ph | 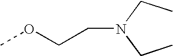 | 501 | E14 |
| E19 | bond | p | Me | H | MeO | 4-Me—Ph | 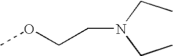 | 447 | E14 |
| E20 | bond | p | Me | H | MeO | 4-MeO—Ph | 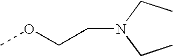 | 463 | E14 |
| E21 | bond | p | Me | H | MeO | 4-Cl—Ph | 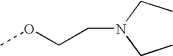 | 468, 470 | E14 |
| E22 | bond | p | Me | H | MeO | 4-Et—Ph | 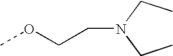 | 461 | E14 |
| E23 | bond | p | Me | H | MeO | 4tBu—Ph | 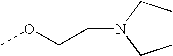 | 489 | E14 |
| E24 | bond | p | Me | F | MeO | Ph | 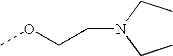 | 451 | E24 |
| E25 | bond | p | Et | H | Me | Ph | 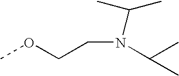 | 459 | E25 |
| E26 | bond | p | Et | H | Me | Cy | 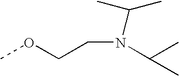 | 465 | E25 |
| E27 | CH₂ | P | Et | H | Me | Ph | 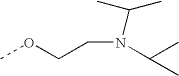 | 473 | E25 |

TABLE F
Encompassing compounds of general formula (VII) a subset of
formula (1) where A = H and OMe, X = O, R4 = 3-pyridyl,
R5 = phenyl, Z = a para bond

| | |
|---|---|
| | (VII) |
| 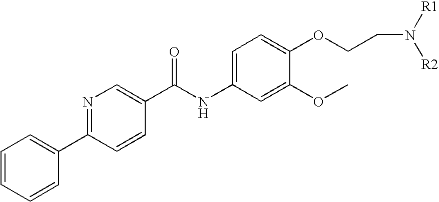 | |

| Example No. | 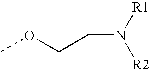 | [M + H]⁺ | Method |
|---|---|---|---|
| F1 | R1 = R2 = Me | 392 | F1 |
| F2 | 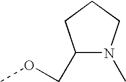 | 418 | F1 |
| F3 | 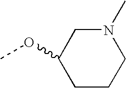 | 418 | F1 |
| F4 | 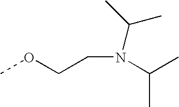 | 448 | F1 |

TABLE G
Encompassing compounds of general formula (VIII) a subset of formula (I)
where A = H and OMe, R3 = H, X = O; R4 = phenyl, Z = O, CH₂ or a bond and
R5 = Ph or cyclohexyl (Cy), Y is a chain of 3 or 4 carbon atoms optionally
substituted by an hydroxyl group.

(VIII)

| Example No. | Z | m/ p | R5 | XYN | 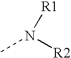 | [M + H]⁺ | method |
|---|---|---|---|---|---|---|---|
| G1 | bond | p | Ph | 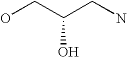 | 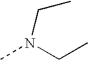 | 449 | G1 |
| G2 | bond | p | Ph | 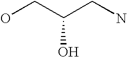 | 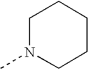 | 461 | G1 |
| G3 | bond | p | Ph | 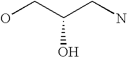 | 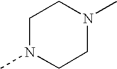 | 476 | G1 |
| G4 | bond | p | Ph | 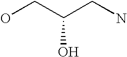 | 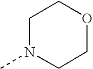 | 476 | G1 |
| G5 | bond | p | Ph | 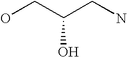 | 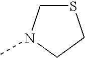 | 465 | G5 |
| G6 | bond | p | Ph | 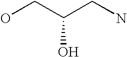 | 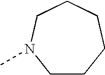 | 475 | G1 |
| G7 | bond | p | Ph | 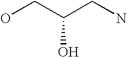 | 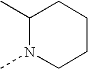 | 475 | G1 |
| G8 | bond | p | Cy | 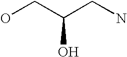 | 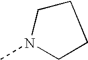 | 453 | G8 |
| G9 | bond | p | Ph | 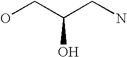 | 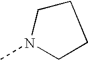 | 447 | G8 |
| G10 | bond | p | Cy | 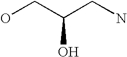 | 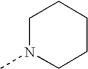 | 455 | G8 |
| G11 | bond | p | Ph | 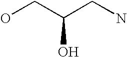 | 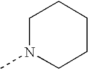 | 449 | G8 |
| G12 | bond | p | Cy | 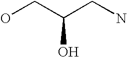 | 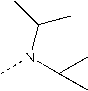 | 483 | G5,G8 |
| G13 | bond | p | Ph | 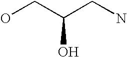 | 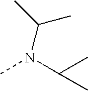 | 477 | G5,G8 |
| G14 | bond | p | Cy | 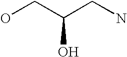 | 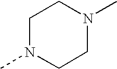 | 482 | G8 |
| G15 | bond | p | Ph | 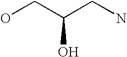 | 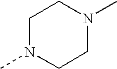 | 476 | G8 |
| G16 | bond | p | Cy | 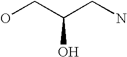 | 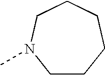 | 481 | G8 |
| G17 | bond | p | Cy | 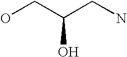 | 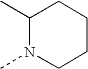 | 481 | G8 |
| G18 | bond | p | Ph | 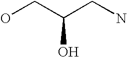 | 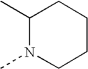 | 475 | G8 |
| G19 | bond | p | Ph | 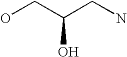 | 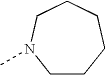 | 475 | G8 |
| G20 | bond | p | Ph | 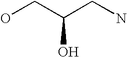 | 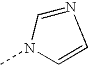 | 444 | G8,G5 |
| G21 | bond | p | Ph | 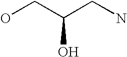 | 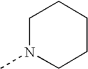 | 461 | G8 |
| G22 | bond | P | Ph | 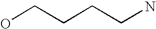 | NMe₂ | 419 | G22 |
| G23 | O | m | Ph | 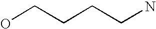 | NMe₂ | 435 | G22 |
| G24 | CH₂ | p | Ph | 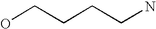 | NMe₂ | 433 | G22 |
| G25 | bond | p | Cy | 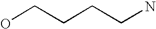 | NMe₂ | 425 | G22 |

TABLE H
Encompassing compounds of general formula (IX) a subset of
formula (I) where A = H and OMe, R3 = H, X = N; R4 = phenyl,
Z = a para substituted bond and R5 = Ph or cyclohexyl (Cy),
Y and R2 form a piperazinyl ring between X and N.

| | |
|---|---|
| | (IX) |
| 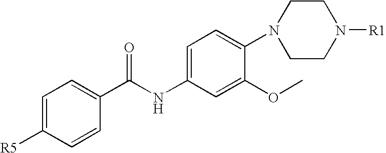 | |

| Example | | | | |
|---|---|---|---|---|
| No. | R5 | R1 | [M + H]⁺ | Method |
| H1 | Cy | Me | 408 | H1 |
| H2 | Cy | Et | 436 | H1 |
| H3 | Cy | iPr | 422 | H1 |

The activity of the compounds used in this invention may be assessed by competitive binding assays to 11CBy receptors, as follows:

Radioligand Binding Studies

Radioligand binding assays were carried out on well washed membranes from HEK293 cells stably expressing 11CBy receptors. Membranes (5-15 mg protein) were incubated with [125I]-Melanin Concentrating Hormone (0.22 nM)(obtained from NEN) in the presence and absence of competing test compounds for 45 min at 37° C. in a buffer (pH 7.4), containing 50 mM Tris and 0.2% BSA. Non-specific binding was defined using 0.1 mM Melanin Concentrating Hormone (obtained from Bachem). The test compounds were added at concentrations between 10M and 10 pM in 10 concentration steps. Following incubation, the reaction was stopped by filtration through GF/B filters and washed with 4×1 ml of ice-cold 50 mM Tris buffer. Microscint 20 (Packard) was added to the filters and the radioactivity measured using a Packard TopCount.

Bound cpm in the presence of test compound was expressed as a fraction of the bound cpm in the absence of test compound and plotted against the concentration of compound. From this an IC50 was determined from which the pKi was calculated.

The most potent compounds of the present invention have pKi values in the range of 7.1 to 7.8 For example:

| Example | pKi range |
|---|---|
| A48 | 7.5-7.8 |
| B2 | 7.1-7.4 |
| C8 | 7.1-7.4 |
| D15 | 7.5-7.8 |
| E9 | 7.5-7.8 |
| F4 | 7.1-7.4 |
| G1 | 7.1-7.4 |
| H1 | 7.1-7.4 |

1. A method of treating the Disorders which comprises administering to a mammal suffering from one or more of the Disorders an effective amount of a compound of formula (I), or a pharmaceutically acceptable salt or solvate thereof, in which: 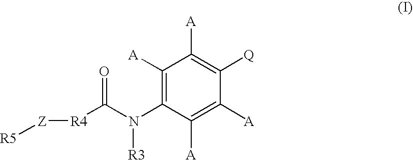
each A is independently hydrogen, a C₁₋₆ alkyl optionally substituted by hydroxyl, C₁₋₆ alkoxy, C₁₋₆ alkenyl or C₁₋₆ acyl group or a halogen atom or hydroxyl, CN or CF₃ group;
R3 is hydrogen, methyl or ethyl;
R4 is an optionally substituted aromatic carbocyclic or heterocyclic ring;
Z is an O or S atom, or an NH or CH₂ group, or a single bond, at the 3 or 4 position of R4 relative to the carbonyl group;
R5 is an optionally substituted aromatic carbocyclic or heterocyclic ring, or an optionally substituted, saturated or unsaturated, carbocyclic or heterocyclic ring;
and Q is 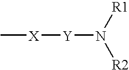
(a) where X is an O or S atom;
Y is a linear or branched C₂₋₄ alkylene group, optionally substituted by a hydroxyl group, or is a C₅₋₆ cycloalkylene group,
R1 and R2 are independently a linear or branched C₁₋₆ alkyl, phenyl C₁₋₆ alkyl group; or
(b) where X is an O or S atom;
Y is a linear or branched C₂₋₄ alkylene group, optionally substituted by a hydroxyl group, R1 and R2 are linked to form a 5, 6 or 7-membered ring optionally containing one or more further heteroatom selected from O, S or N, where N or C ring atoms are optionally substituted by Ra, —CO-Ra, —CO—NH-Ra, or CO—O-Ra, where Ra is a linear or branched C₁₋₆ allyl or aryl group; and the 5, 6 or 7-membered ring is optionally fused to an optionally substituted benzene ring, or a ring atom of the 5, 6 or 7-membered ring is optionally liked by a single bond or methylene group to Y; or
(c) where X is an O or S atom,
Y is a C₂₋₄ alkylene group, R1 is a C₂₋₄ alkylene group linked to Y to form a 5 or 6 membered ring and R2 is a linear or branched C₁₋₆ alkyl group; or
(d) where X is a N atom,
Y is a C₂₋₄ alkylene group, R1 is a C₂₋₄ alkylene group linked to X to form a 5 or 6 membered ring and R2 is a linear or branched C₁₋₆ alkyl group.

2. A compound of formula (I) as defined in claim 1, or a salt or solvate thereof, in which R3 is methyl or ethyl.

3. A compound according to claim 2, which is any one of the compounds set out in Table E herein.

4. A compound of formula (I) as defined in claim 1 or a salt or solvate thereof, excluding the compounds:
N-[4-[2-[bis(1-methylethyl)anmino]ethoxy]-2-fluorophenyl]-[1,1′-biphenyl]-4-carboxamide,
N-[4-[2-[bis(1-methylethyl)amino]ethoxy]phenyl]-[1,1′-biphenyl]-4-carboxamide, biphenyl-4-carboxylic acid [4-(2-diisopropylamino-ethoxy)-phenyl]-amide,
N-[4-(2-diisopropylamino-ethoxy)-phenyl]-4-phenoxy-benzamide,
N-[4-(2-diethylamino-ethoxy)-phenyl]-4-phenoxy-benzamide,
N-[4-(2-diisopropylamino-ethoxy)-phenyl]-3-phenoxy-benzamide
N-[4-(2-diethylamino-ethoxy)-phenyl]-3-phenoxy-benzamide,
4-cyclohexyl-N-[4-(2-diisopropylamino-ethoxy)-phenyl]-benzamide,
4-cyclohexyl-N-[4-(2-diethylamino-ethoxy)-phenyl]-benzamide,
4-benzyl-N-[4-(2-diisopropylamino-ethoxy)-phenyl]-benzamide,
4-benzyl-N-[4-(2-diethylamino-ethoxy)-phenyl]-benzamide,
4′-ethyl-biphenyl-4-carboxylic acid [4-(2-diisopropylamino-ethoxy)-phenyl]-amide,
and 4′-ethyl-biphenyl-4-carboxylic acid [4-(2-diethylamino-ethoxy)-phenyl]-amide.

5. A process for the preparation of a compound of formula (I), or a salt or solvate thereof, as defined in claim 2, which process comprises the reaction of a compound of formula (X)
R5-Z-R4-COL (X)
where R5, Z, and R4 are as defined for formula (I) in claim 1, and L is a leaving group with a compound of formula (XI) 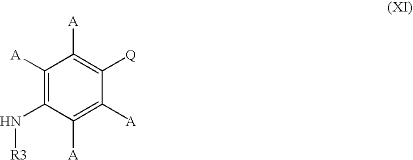
wherein Q and A are as defined in formula (I) in claim 1 and R3 is methyl or ethyl.

6. A process for the preparation of a compound of formula (I), or a salt or solvate thereof, as defined in claim 1, which process comprises the reaction of a compound of formula (X) wherein R5, Z, and R4 are as defined for formula (I) in claim 1 with a compound of formula (XI) wherein Q, A, and R3 are as defined in claim 1, with the proviso that a process for the preparation of:
N-[4-[2-[bis(1-methylethyl)amino]ethoxy]-2-fluorophenyl]-[1,1′-biphenyl]-4-carboxamide,
N-[4-[2-[bis(1-methylethyl)amino]ethoxy]phenyl]-[1,1′-biphenyl]4-carboxamide, biphenyl-4-carboxylic acid [4-(2-diisopropylamino-ethoxy)-phenyl]-amide,
N-[4-(2-diisopropylamino-ethoxy)-phenyl]-4-phenoxy-benzamide,
N-[4-(2-diethylamino-ethoxy)-phenyl]-4-phenoxy-benzamide,
N-[4-(2-diisopropylamino-ethoxy)-phenyl]-3-phenoxy-benzamide,
N-[4-(2-diethylamino-ethoxy)-phenyl]-3-phenoxy-benzamide,
4-cyclohexyl-N-[4-(2-diisopropylamino-ethoxy)-phenyl]-benzamide,
4-cyclohexyl-N-[4-(2-diethylamino-ethoxy)-phenyl]-benzamide,
4-benzyl-N-[4-(2-diisopropylamino-ethoxy)-phenyl]-benzamide,
4-benzyl-N-[4-(2-diethylamino-ethoxy)-phenyl]-benzamide,
4′-ethyl-biphenyl-4-carboxylic acid [4-(2-diisopropylamino-ethoxy)-phenyl]-amide,
and 4′-ethyl-biphenyl-4-carboxylic acid [4-(2-diethylamino-ethoxy)-phenyl]-amide is excluded.

7. A pharmaceutical composition for use in the treatment and/or prophylaxis of one or more of the Disorders which comprises a compound of this invention, or a pharmaceutically acceptable salt or solvate thereof, and a pharmaceutically acceptable carrier.

8. A method of treatment and/or prophylaxis of one or more of the Disorders comprising administering to the sufferer in need thereof an effective or prophylactic amount of a compound of this invention, or a pharmaceutically acceptable salt or solvate thereof.

9. Use of a compound of this invention, or a pharmaceutically acceptable salt or solvate thereof, for the manufacture of a medicament for the treatment and/or prophylaxis of one or more of the Disorders.

10. Use of a novel compound of this invention, or a pharmaceutically acceptable salt or solvate, thereof as a therapeutic agent, in particular for the treatment and/or prophylaxis of one or more of the Disorders.

11. A method for the treatment of diabetes, major depression, manic depression, anxiety, schizophrenia and sleep disorders, in human or non-human mammals which method comprises the administration of a therapeutically effective amount of an antagonist to the human 11CBy receptor.
